SEQUENCE LISTING (4 sequences)

SEQ ID NO: 1 - DNA, 1407 bp, Triticum aestivum
atgtcctcctccacggggagcttggaccacgcagggttcacgttcacgccgccgcccttcatcacgtccttcaccgagcttctgtcagggtccggcgccggcgacgcggagcggtcgccaagggggttcaaccgagggggccgggccggggcgcccaagttcaagtccgcgcagccgcccagcctgcccatctcgtcgcccttctcctgcttctccatccctgcgggcctcagccctgctgagctgctcgactcccccgttctcctcaactactcccatatcctggcatctccgaccacaggtgcgatccctgcgcggaggtacgactggcaggcgagcgccgatctgaacacctttcagcaggatgagccctgccgaggagacagcggcctctttggcttctcctttcacgcagtgaagtccaacgccacggtcaacgctcaagcaaattgcttacctttattcaaggagcagcagcagcagcaacaacaacaagtggtagaagtgagcaacaagagcagcagcggcggcggcaacaacaagcaggttgaggacggatacaactggaggaagtacgggcagaagcaagttaaggggagcgagaacccgcggagctactacaagtgcacctacaacaattgctccatgaagaagaaagtggagcgctctctcgccgacggccgcatcacgcagattgtctacaagggcgcacacgaccacccgaagcccctctccacgcgccgcaactcctccggctgtgcggcggtcgtcgcggaggatcatgccaacggctcggagcactctgggccgacgccggagaattcatccgtcactttcggagacgatgaggccgacaacgggttgcagctcagtgatggcgctgagcccgtggccaagcgccggaaggagcatgccgacaacgagggcagttcaggtggcaccggcggctgcgggaagcccgtgcgcgagccgaggcttgtggtgcagacgctgagcgatatagacatactcgacgacggcttccggtggaggaagtacgggcagaaggttgtcaagggcaatcccaaccccaggagctactacaagtgcacaacggtgggttgcccagtgcgcaagcatgtggagcgggccgcgcacgacaaccgcgcggtgatcaccacctacgagggtaagcacaaccacgacatgccggtcggccggggtgccggtgccagccgggcgctgccgacgtcatcttcctcggacagctcggtcgtaacctggcctgccgccgtgcaggccccgtacaccctcgagatgctcaccaaccctgccgccggccaccgaggctatgcagccggcggcgccttccagcgcaccaaggacgagccccgggacgacatgttcgtcgagtcgctcctctgctag

SEQ ID NO: 2 - protein, 468 aa, Triticum aestivum
MSSSTGSLDHAGFTFTPPPFITSFTELLSGSGAGDAERSPRGFNRGGRAGAPKFKSAQPPSLPISSPFSCFSIPAGLSPAELLDSPVLLNYSHILASPTTGAIPARRYDWQASADLNTFQQDEPCRGDSGLFGFSFHAVKSNATVNAQANCLPLFKEQQQQQQQQVVEVSNKSSSGGGNNKQVEDGYNWRKYGQKQVKGSENPRSYYKCTYNNCSMKKKVERSLADGRITQIVYKGAHDHPKPLSTRRNSSGCAAVVAEDHANGSEHSGPTPENSSVTFGDDEADNGLQLSDGAEPVAKRRKEHADNEGSSGGTGGCGKPVREPRLVVQTLSDIDILDDGFRWRKYGQKVVKGNPNPRSYYKCTTVGCPVRKHVERAAHDNRAVITTYEGKHNHDMPVGRGAGASRALPTSSSSDSSVVTWPAAVQAPYTLEMLTNPAAGHRGYAAGGAFQRTKDEPRDDMFVESLLC

SEQ ID NO: 3 - DNA, 1407 bp, Triticum aestivum
atggcggcggggcagtggtcaggcatcggcgacggcggcggcctctgggccccgcccgcgctcgacagcctcttccccgacgaccagccgtcgccggccgcctcggcgctgggcttcttcggtggatccctcgcgcagctcccttcccctccgccgctctgcggcaccgcgctcctcgggtacccccaggacaactttgatgtgttccatgaacgagacctagcacagctggcagcacaagtggctcaaaagaaagagttgcgggaaaaacaaggggcgggattgcatcacaagattggacctcaactagctttttctaaatacagtatacttgatcaagtggacaactcctcttctttctcattggcaacttcagtgctgacacctcagcatgtcagttcttccgtaggcgcggcattaatgcagggacggactttgccatcacacactggtagtggtagtgtcaacactggaccaactggagttttacaagcgctccaagattcatccaccactctggacagtatcaacactggatcaactggagttctggaagcactccaaggttcatccatcactctggatagacctgctgatgatggatacaactggcgtaagtatggacaaaaggcagtcaagggtgggaagtatccaaggagctattacaaatgtaccctgaattgcccggccaggaaaaatgtagagcactctgcagatagacgaattattaaaataatttatagaggtcagcactgccatgaacccccctcaaagaggtttaaagattgtggtgatttattgaatgagttaaatgatttcgatgatgccaaggagccttcaactaaatcacaattaggttgtcaaggttattatggaaaacctataacgccaaatggaatgatgacggatgttttattgccaacgaaggaagagggggatgagcaattatctagtttaagtgatatccgggaaggtgatggtgaaataagaactgttgatggagatgatggtgatgccgatgcaaatgaaaggaatgcaccaggtcaaaagattatcgtgagtacaacgagcgatgctgatcttttggacgacggctataggtggcgcaagtatggacagaaagtggtgagaggaaatcctcacccaaggagctattacaagtgcacttaccaaggatgcgacgtcaagaagcatatcgagagatcttccgaggaaccacatgctgtgataactacatacgaagggaagcatacgcatgacgtgcctgagtctaggaacagaagccaagccacaggtcaacaccactgcaaagagcagacttattcagaacaatcagctgcaagcttctgcagtagctcggaaaagagaaaatatggaacagccattctgaacgatctcgccttctag

SEQ ID NO: 4 - protein, 468 aa, Triticum aestivum
MAAGQWSGIGDGGGLWAPPALDSLFPDDQPSPAASALGFFGGSLAQLPSPPPLCGTALLGYPQDNFDVFHERDLAQLAAQVAQKKELREKQGAGLHHKIGPQLAFSKYSILDQVDNSSSFSLATSVLTPQHVSSSVGAALMQGRTLPSHTGSGSVNTGPTGVLQALQDSSTTLDSINTGSTGVLEALQGSSITLDRPADDGYNWRKYGQKAVKGGKYPRSYYKCTLNCPARKNVEHSADRRIIKIIYRGQHCHEPPSKRFKDCGDLLNELNDFDDAKEPSTKSQLGCQGYYGKPITPNGMMTDVLLPTKEEGDEQLSSLSDIREGDGEIRTVDGDDGDADANERNAPGQKIIVSTTSDADLLDDGYRWRKYGQKVVRGNPHPRSYYKCTYQGCDVKKHIERSSEEPHAVITTYEGKHTHDVPESRNRSQATGQHHCKEQTYSEQSAASFCSSSEKRKYGTAILNDLAF

(19) United States
(12) Patent Application Publication
(10) Pub. No.: US 2013/0014289 A1
(43) Pub. Date: Jan. 10, 2013

(54) GENES CONFERRING STRESS TOLERANCE IN PLANTS AND USES THEREOF

(75) Inventors: Jinsong Zhang, Beijing (CN); Shouyi Chen, Beijing (CN); Canfang Niu, Beijing (CN); Biao Ma, Beijing (CN); Wanke Zhang, Beijing (CN); Qing Lin, Beijing (CN); Sijie He, Beijing (CN)

(73) Assignee: Institute of Genetics and Developmental Biology, Chinese Academy of Sciences, Bejing (CN)

(21) Appl. No.: 13/580,935

(22) PCT Filed: Feb. 24, 2010
(86) PCT No.: PCT/CN10/70736

§ 371 Date: Sep. 10, 2012

(51) Int. Cl. A01H 5/00; A01H 1/02; A01H 5/10; C12N 15/82

(52) U.S. Cl. 800/260; 800/298; 800/320.1; 800/320.3; 800/320; 800/317.2; 800/313; 800/305; 800/310; 800/317.4; 800/312; 800/306; 800/317.3; 800/314; 800/278; 800/289; 800/320.2; 800/317.1; 800/307; 800/317; 800/309; 800/318; 800/322

(57) ABSTRACT

Compositions and methods for imparting stress tolerance to plants using WRKY nucleic acid and polypeptides.

### FIELD OF THE INVENTION

The invention relates generally to compositions and methods for conferring stress tolerance to plants, including polynucleotides, polypeptides, vectors and host cells. The present invention also relates generally to plants transformed by the aforementioned compositions and methods.

### BACKGROUND OF THE INVENTION

Most crop species throughout the world are exposed to various abiotic stresses that cause adverse effects on their growth and productivity, and drought, salt and low temperatures are the most significant. Salt stress disturbs ionic and osmotic homeostasis, while low temperature leads to mechanical damage, changes in activities of macromolecules, and reduced osmotic potential in cells.

Upon exposure to abiotic stress(es), plants activate diverse genes that are involved in stress tolerance signaling. These stress-induced genes can be divided into effector proteins and regulatory proteins, the latter of which include transcription factors. Alteration in the expression of transcription factor genes normally results in dramatic changes in plants, and overexpression of a number of transcription factor genes confer stress tolerance in transgenic Arabidopsis plants. Accordingly, identification and genetic manipulation of various transcription factors has received much attention.

One of the families of transcription factors that have been identified and studied is the WRKY family, which is named after the WRKY domain common to its members. This domain is about 60 amino acids in length and contains a highly-conserved WRKYGQK heptapeptide at its N-terminus with a zinc-finger-like motif at its C-terminus. The WRKY family contains 74 members in Arabidopsis and more than 100 members in rice. WRKY genes have also been identified in many other species, including higher plants such as sweet potato (Ipomoea batatas), barley (Hordeum vulgare), creosote bush (Larrea tridentata) and soybean (Glycine max), lower plants such as fern (Ceratopteris richardii) and moss (Physcomitrella patens), and nonplant species such as green algae (Chlamydomonas reinhardtii), diplomonads (Giardia lamblia) and amoebozoa (Dictyostelium discoideum).

All identified WRKY proteins contain one or two WRKY domains. The single WRKY domain-containing protein has more similarities in sequence and DNA-binding activity to the C-terminal than to the N-terminal domain of the two-WRKY-domain-containing proteins. Most of the identified WRKY proteins with a WRKYGQK sequence can bind the W-box (TTGAC[C/T]) to regulate gene expression, while some other WRKY proteins with the mutated WRKYGKK motif can bind the WK-box (TTTTCCAC) but not W-box element. In addition to the WRKY domain, some other WRKY proteins possess nuclear localization signals, leucine zippers, serine-threonine-rich regions, glutamine-rich regions, proline-rich regions, acidic regions or TIR-NBS-LRR domains, and these diverse structures reflect their multifunctional natures.

Many WRKY genes play regulatory roles in responses of plants to salicylic acid and biotic stress, such as pathogenic bacteria, fungi, and viruses. WRKY genes are also reported to play roles in seed development and germination, senescence, and secondary metabolism. Further, accumulating cases demonstrate that WRKY genes are involved in responses to abiotic stresses and ABA signaling in plants. The rice WRKY genes, OsWRKY24, -45, -72 and -77, are involved in ABA signaling. Arabidopsis WRKY genes can be induced by drought and cold stress. Several reports have also shown that WRKY genes responded to abiotic stress and ABA signaling in other plants, and it was recently reported that three soybean WRKY genes conferred abiotic stress tolerance in transgenic Arabdidopsis plants.

Addressing the aforementioned abiotic stresses continues to provide a significant challenge in providing sustainable crop production to feed an ever-growing world population that relies heavily on crop species (e.g., rice, wheat) to meet its dietary needs. As such, there is a continuing need to identify and genetically manipulate additional genes that are involved in abiotic stress tolerance in order to create improved crop cultivars.

### SUMMARY OF THE INVENTION

The present invention relates to isolated wheat WRKY polynucleotides, polypeptides, vectors and host cells expressing isolated WRKY polynucleotides capable of imparting stress tolerance to plants, particularly drought/osmotic stress, salt stress and cold/freezing stress.

The isolated WRKY polynucleotides provided herein include nucleic acids comprising (a) a nucleotide sequence of any one of SEQ ID NOs: 1 and 3; (b) a nucleotide sequence at least 70% identical to (a); (c) a nucleotide sequence that specifically hybridizes to the complement of (a) under stringent hybridization conditions; (d) an open reading frame encoding a WRKY protein comprising a polypeptide sequence of any one of SEQ ID NOs: 2, and 4; (e) an open reading frame encoding a WRKY protein comprising a polypeptide sequence at least 70% identical to (d) and possessing at least one WRKY domain; and (f) a nucleotide sequence that is the complement of any one of (a)-(e).

The isolated WRKY polypeptides provided herein include (a) an amino acid sequence of any one of SEQ ID NOs: 2 and 4; and (b) an amino acid sequence at least 70% identical to (a) that comprises at least one WRY domain.

The host cells provided herein include those comprising the isolated polynucleotides and vectors of the present invention. The host cell can be from an animal, plant, or microorganism, such as E. coli. Plant cells are particularly contemplated. The host cell can be isolated, excised, or cultivated. The host cell may also be part of a plant.

The present invention further relates to a plant or a part of a plant that comprises a host cell of the present invention. Monocots such as such as wheat, barley, rice, maize, sorghum, oats, and rye are particularly contemplated. The present invention also relates to the transgenic seeds of the plants.

The present invention further relates to a method for producing a plant comprising regenerating a transgenic plant from a host cell of the present invention, or hybridizing a transgenic plant of the present invention to another non-transgenic plant. Plants produced by these methods are also encompassed by the present invention, and plants having improved stress tolerance to drought/osmotic stress, salt stress and cold/freezing stress are particularly contemplated, as are crop plants, such as wheat, barley, rice, maize, sorghum, oats, and rye.

The present invention further relates to methods of altering a trait in a plant or part of a plant using the isolated polynucleotides, polypeptides, constructs and vectors of the present invention. These traits include tolerance to drought/osmotic stress, salt stress and cold/freezing stress. Preferably the aforementioned traits are altered so that they are increased or otherwise improved. In one embodiment, one or more traits of a plant are altered by expressing in a plant an isolated nucleic acid such as (a) a nucleotide sequence of any one of SEQ ID NOs: 1 and 3; (b) a nucleotide sequence at least 70% identical to (a); (c) a nucleotide sequence that specifically hybridizes to the complement of (a) under stringent hybridization conditions; (d) an open reading frame encoding a WRKY protein comprising a polypeptide sequence of any one of SEQ ID NOs: 2 and 4; (e) an open reading frame encoding a WRKY protein comprising a polypeptide sequence at least 70% identical to (d) and possessing at least one WRKY domain; and (f) a nucleotide sequence that is the complement of any one of (a)-(e). In another embodiment, one or more traits of a plant are altered by expressing in a plant an isolated hypermorphic WRKY allele. In another embodiment, one or more traits of a plant are altered by increasing the expression of a WRKY nucleic acid or polypeptide in the plant. In yet another embodiment, one or more traits of a plant are altered by altering the function of a WRKY polypeptide in the plant.

The present invention further relates to plants, plant parts and transgenic seeds created through the aforementioned methods of altering a trait in a plant. Such contemplated plants, plant parts and transgenic seeds may be created directly from the aforementioned methods. Alternatively, the contemplated plants, plant parts and transgenic seeds may be derived from a host cell (e.g., regenerated from a host cell) or produced by crossing a transgenic plant with one or more altered traits with a non-transgenic plant.

The present invention further relates to methods of altering the expression of a stress-related gene selected from the group consisting of DREB2A, RD29A, RD29, COR15A, STZ and COR6.6. In certain embodiments, the expression of one or more of these stress-related genes is increased by increasing the expression one or more WRKY polypeptide in a host cell, plant or plant part. In other embodiments, the expression of one or more of these stress-related genes is decreased by decreasing the expression of one or more WRKY polypeptides in a host cell, plant or plant part.

The present invention further relates to methods of identifying WRKY binding agents and inhibitors. In one embodiment, the method comprises (a) providing an isolated WRKY protein; (b) contacting the isolated WRKY protein with an agent under conditions sufficient for binding; (c) assaying binding of the agent to the isolated WRKY protein; and (d) selecting an agent that demonstrates specific binding to the isolated WRKY protein. In another embodiment, the method comprises (a) providing a host cell expressing a WRKY protein; (b) contacting the host cell with an agent; (c) assaying expression of WRKY protein; and (d) selecting an agent that induces altered expression of WRKY protein. In yet another embodiment, the method comprises (a) providing a plant or part of a plant expressing a WRKY protein; (b) contacting the plant or the part of the plant with an agent; (c) assaying for alteration of a trait of the plant or the part of the plant; and (d) selecting an agent that alters the trait. The traits to be assayed are those known to be affected by WRKY expression (e.g., drought/osmotic stress tolerance, salt tolerance, and cold/freezing tolerance). Preferably agents that increase or otherwise improve these traits are selected. However, agents that negatively impact a trait are contemplated as well.

The present invention also relates to methods of inhibiting WRKY in a plant using the binding agents and inhibitors identified by the methods herein.

### BRIEF DESCRIPTION OF THE DRAWINGS

FIG. 1 shows the expression of the TaWRKY genes in response to various stresses and ABA treatment. (a) Expression of TaWRKY2 and -19 genes in 15-day-old wheat (cultivar Xifeng 20, drought-resistant) seedlings treated with drought, 250 mM NaCl, 4° C. or wounding as revealed by RT-PCR. Taactin gene was amplified as an internal control. (b) Expression of the TaWRKY genes in wheat seedlings treated with 100 μM ABA. Others are as in (a). (c) Expression of the TaWRKY genes in different organs of 20-day-old wheat seedlings as revealed by RT-PCR. Taactin gene was amplified as an internal control.

FIG. 2 shows the osmotic (drought) stress tolerance of the TaWRKY2-overexpressing plants. (a) Expression of TaWRKY2 in different transgenic lines. (b) Growth comparison of the three transgenic lines (2-12, 2-1, and 2-2) with the Col-0 plants under sorbitol treatment. Seven-day-old plants from MS plate were transfered on 0.5×MS containing 300 mM sorbitol and maintained for 20 days. (c) Growth of the plants under normal condition (CK) and after osmotic stress. Seedlings from (b) were transfered in pots and maintained for 20 days. (d) Survival rates of the plants in (c) after stress recovery. (e) Bolting rate of the plants in (c) after stress recovery. (f) Comparison of plant heights after stress recovery in (c). For (d), (e) and (f), values are the average of three repeated experiments (36 plants for each experiment) SD. Asterisks indicate significant differences between transgenic plants and the Col-0 (*P<0.05; **P<0.01). (g) Soluble sugar contents in plants after sorbitol treatment. (h) Relative electrolyte leakage in leaves from plants after sorbitol treatment. Seven-day-old seedlings were transferred onto 0.5×MS plate containing sorbitol and maintained for 5 days. For (g) and (h), each data point represents means from three replicates ±SD from one representative of three repeated experiments. Asterisks indicate significant differences between the transgenic plants and the Col-0 plants (*P<0.05; **P<0.01).

FIG. 3 shows the salt stress tolerance of the Ta WRKY2-overexpressing plants. (a) Phenotype of the NaCl-treated transgenic lines (2-12, 2-1, and 2-2) and Col-0. (b) Growth recovery of the plants from (a) in pots for 10 days. (c) Growth recovery of the plants from (a) in pots for 25 days. (d) Survival rate of the plants from (b). (e) Bolting rate of the plants from (c). (f) Plant heights of the Col-0 and transgenic plants in (c). For (d), (e) and (f), values indicate the average of three repeated experiments (36 plants for each experiment)±SD. (g) Soluble sugar contents in plants after salt stress. (h) MDA contents in plants after salt stress. (i) Relative electrolyte leakage in leaves from salt-treated plants. For (g), (h) and (i), seven-day-old seedlings were transferred onto 0.5×MS plate containing NaCl and maintained for 5 days. Each data point is means from 3 replicates SD from one representative of 3 repeated experiments. From (d) to (i), asterisks indicate significant differences between the transgenic plants and the Col-0 plants (*P<0.05; **P<0.01).

FIG. 4 shows the osmotic (drought) stress tolerance of the TaWRKY19-overexpressing plants. (a) Expression of TaWRKY19 in Col-0 and transgenic lines. (b) Growth comparison of the transgenic lines (19-1, 19-4, and 19-5) with the Col-0 plants under sorbitol treatment. (c) Growth recovery of sorbitol-stressed seedlings in pots. (d) Survival rate of the plants under normal (CK) or stress conditions. (e) Comparison of plant bolting rate. (f) Comparison of plant heights after sorbitol treatment. (g) Relative electrolyte leakage of leaves from sorbitol-treated Col-0 and transgenic plants. Seven-day-old seedlings were transferred onto 0.5×MS plate containing 300 mM sorbitol and maintained for 5 days, and then used for analysis in (g). Others are as in FIG. 3.

FIG. 5 shows the performance of the TaWRKY19-overexpressing plants under salt stress. (a) Phenotypic comparison of NaCl-treated Col-0 and transgenic lines (19-1, 19-4, and 19-5). (b) Growth of the salt-stressed plants after 10 d recovery. (c) Growth of the salt-treated plants after recovery for 25 days. (d) Survival rate of the salt-stressed plants after recovery for 10 d. (e) Bolting rate of the salt-stressed plants after 25 days recovery. (f) Plant heights of the salt-stressed plants after 25 days recovery. (g) Soluble sugar contents in salt-treated plants. (h) MDA contents in salt-treated plants. (i) Relative electrolyte leakage of leaves from salt-treated plants. Others are as in FIG. 2. Asterisks indicate significant differences between transgenic plants and the Col-0 (*P<0.05; **P<0.01).

FIG. 6 shows the freezing tolerance of the TaWRKY19-overexpressing plants. (a) Phenotype comparison of freezing-treated Col-0 and transgenic lines (19-1, 19-4, and 19-5). (b) Survival rate of the freezing-treated plants. (c) Bolting rate of the plants after recovery from freezing. (d) Plant heights of the Col-0 and transgenic plants after recovery from freezing. (e) Soluble sugar contents in plants under normal condition (CK) and after freezing. (f) MDA contents in plants under normal condition and after freezing. (g) Relative electrolyte leakage in leaves from freezing-treated Col-0 and transgenic lines. Others are as in FIG. 2. Asterisks indicate significant differences between the transgenic plants and the Col-0 plants (*P<0.05; **P<0.01).

FIG. 7 shows the subcellular localization and DNA-binding ability of TaWRKY proteins. (a) Subcellular localization of TaWRKY proteins in Arabidopsis protoplasts. GFP protein alone (CK) or TaWRKY-GFP fusions were expressed transiently under the control of CaMV 35S promoter in Arabidopsis protoplasts. The photographs are dark field (left) for green fluorescence and bright field (right) for the morphology of the cells. Arrows point to nucleus. (b) SDS-PAGE of the MBP-fused TaWRKY proteins. The sizes of the protein markers were indicated on the left. Arrows indicate the corresponding protein bands. (c) DNA-binding ability of the TaWRKY proteins. The proteins were incubated with γ-32P-labeled W box (Wb) or mutated W box (mWb) elements in the presence (+) or absence (−) of 10-fold molar excess of unlabelled competitors. The protein/DNA complexes were indicated by arrows. Both the Wb and mWb were in triple tandem repeats. The W box core sequence was underlined and asterisks indicate the mutated bases in the W box element. MBP was used as a negative control.

FIG. 8 shows the transcriptional activation and transactivation ability of the TaWRKY proteins. (a) Transcriptional activation of TaWRKY2 and TaWRKY19 proteins. The TaWRKY genes were fused with GAL4 DBD to generate pBD-TaWRKYs. The pBD was negative control and the pGAL4 positive control. Growth of the yeast cells harboring a tested gene on SD/-His and blue color in the presence of X-gal in β-galactosidase assay indicate that the corresponding protein has transcriptional activation abiltity. All the transformants grew well on YPAD under normal conditions. ‘+’ indicates that the protein has transactivation ability and ‘−’ indicates that the protein has no such ability. (b) Transactivation activity of the TaWRKY proteins in Arabidopsis protoplasts by a dual-luciferase reporter assay. GAL4 DBD is negative control and the VP16 is positive control. Asterisks indicate significant differences compared to the negative control GAL4 DBD (*P<0.05; **P<0.01).

FIG. 9 shows the expression of stress-related genes in Ta WRKY-transgenic plants. (a) Expression of STZ and RD29B in Ta WRKY2-overexpressing plants (2-12, 2-1 and 2-2). (b) Expression of DREB2A, RD29A, RD29B and Cor6.6 in TaWRKY19-overexpressing plants (19-1, 19-4 and 19-5). ‘rRNA’ indicates 18S and 28S rRNA as a loading control.

FIG. 10 shows the binding of the TaWRKY proteins to cis-DNA elements in the promoter regions of downstream genes. (a) Distribution of various W box elements in the 1.5 kb promoter regions of the six Ta WRKY-regulated genes. (b) Putative W boxes used for DNA-binding analysis. STZ-1 and STZ-2 fragments indicate DNA sequences in STZ promoter region at position −1131 to −1168 and position −334 to −303, respectively. One to three predicted W boxes (underlined) were included in sequences from promoter regions of Cor15A, Cor6.6, RD29A and RD29B. (c) TaWRKY proteins bind to the cis-DNA elements in promoter regions of downstream genes. The TaWRKY proteins were incubated with [γ-32P]-dATP-labeled probes in the presence (+) or absence (−) of 10-fold molar excess of non-labeled competitors, and the specific [γ-32P]-labeled DNA/protein complexes were indicated by arrows.

### DETAILED DESCRIPTION OF THE INVENTION

### WRKY Nucleic Acids and Proteins

As used herein, the terms “nucleic acid”, “polynucleotide”, “polynucleotide molecule”, “polynucleotide sequence” and plural variants are used interchangeably to refer to a wide variety of molecules, including single strand and double strand DNA and RNA molecules, cDNA sequences, genomic DNA sequences of exons and introns, chemically synthesized DNA and RNA sequences, and sense strands and corresponding antisense strands. Polynucleotides of the invention may also comprise known analogs of natural nucleotides that have similar properties as the reference natural nucleic acid.

As used herein, the terms “polypeptide”, “protein” and plural variants are used interchangeably and refer to a compound made up of a single chain of amino acids joined by peptide bonds. Polypeptides of the invention may comprise naturally occurring amino acids, synthetic amino acids, genetically encoded amino acids, non-genetically encoded amino acids, and combinations thereof. Polypeptides may include both L-form and D-form amino acids.

Representative non-genetically encoded amino acids include but are not limited to 2-aminoadipic acid; 3-aminoadipic acid; β-aminopropionic acid; 2-aminobutyric acid; 4-aminobutyric acid (piperidinic acid); 6-aminocaproic acid; 2-aminoheptanoic acid; 2-aminoisobutyric acid; 3-aminoisobutyric acid; 2-aminopimelic acid; 2,4-diaminobutyric acid; desmosine; 2,2′-diaminopimelic acid; 2,3-diaminopropionic acid; N-ethylglycine; N-ethylasparagine; hydroxylysine; allo-hydroxylysine; 3-hydroxyproline; 4-hydroxyproline; isodesmosine; allo-isoleucine; N-methylglycine (sarcosine); N-methylisoleucine; N-methylvaline; norvaline; norleucine; and ornithine.

Representative derivatized amino acids include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine.

As used herein, the term “isolated” refers to polynucleotides and polypeptides that, but for at least one act of man, do not exist in whatever form or amount they are found. Exemplary embodiments include polynucleotides and polypeptides that are partially, substantially or wholly purified from other molecular species; polynucleotides and polypeptides that are heterologous to a particular cell, organism, or part of an organism; polynucleotides and polypeptides that are not heterologous to a particular cell, organism, or part of an organism, but are expressed at an altered level as a result of the at least one act of man; and polynucleotides and polypeptides that are expressed in the progeny or other downstream products (e.g., fruit) of a cell, organism, or part of an organism subject to the at least one act of man.

Exemplary WRKY polynucleotides of the invention are set forth as SEQ ID NOs: 1 and 3 and substantially identical sequences encoding WRKY proteins capable of altering a trait of a plant, for example, drought/osmotic stress tolerance, salt stress tolerance and cold/freezing stress tolerance.

Exemplary WRKY polypeptides of the invention are set forth as SEQ ID NOs: 2 and 4 and substantially identical proteins capable of altering a trait of a plant, for example, drought/osmotic stress tolerance, salt stress tolerance and cold/freezing stress tolerance.

Substantially identical sequences are those that have at least 60%, preferably at least 80%, preferably at least 85%, more preferably at least 90%, even more preferably at least 95%, and most preferably at least 99% nucleotide or amino acid residue identity, when compared and aligned for maximum correspondence using a sequence comparison algorithm or by visual inspection. Preferably, the substantial identity exists over a region of the sequences that is at least about 50 residues in length, more preferably over a region of at least about 100 residues, and most preferably the sequences are substantially identical over at least about 150 residues. In an especially preferred embodiment, the sequences are substantially identical over the entire length of the coding regions. Furthermore, substantially identical nucleic acids or proteins perform substantially the same function. Substantially identical sequences may be polymorphic sequences, i.e., alternative sequences or alleles in a population. An allelic difference may be as small as one base pair. Substantially identical sequences may also comprise mutagenized sequences, including sequences comprising silent mutations. A mutation may comprise one or more residue changes, a deletion of one or more residues, or an insertion of one or more additional residues. Substantially identical nucleic acids are also identified as nucleic acids that hybridize specifically to or hybridize substantially to a reference sequence (e.g., SEQ ID NO: 1).

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math., 2:482 (1981), by the homology alignment algorithm of Needleman & Wunsch, J. Mol. Biol., 48:443 (1970), by the search for similarity method of Pearson & Lipman, Proc. Natl. Acad. Sci. USA, 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see, Ausubel et al., infra).

One example of an algorithm that is suitable for determining percent sequence identity and sequence similarity is the BLAST algorithm, which is described in Altschul et al., J. Mol. Biol., 215:403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA, 89:10915 (1989)).

In addition to calculating percent sequence identity, the BLAST algorithm also performs a statistical analysis of the similarity between two sequences (see e.g., Karlin & Altschul, Proc. Natl. Acad. Sci. USA, 90:5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

Substantially identical sequences may be polymorphic sequences, i.e., alternative sequences or alleles in a population. An allelic difference may be as small as one base pair. Substantially identical sequences may also comprise mutagenized sequences, including sequences comprising silent mutations. A mutation may comprise one or more residue changes, a deletion of one or more residues, or an insertion of one or more additional residues.

Another indication that two nucleic acid sequences are substantially identical is that the two molecules hybridize to each other under stringent conditions. Stringent conditions are those under which a nucleic acid probe will typically hybridize to its target sequence but to no other sequences when that sequence is present in a complex nucleic acid mixture (e.g., total cellular DNA or RNA). Stringent hybridization conditions and stringent hybridization wash conditions in the context of nucleic acid hybridization experiments such as Southern and Northern blot analyses are both sequence- and environment-dependent. An extensive guide to the hybridization of nucleic acids is found in Tijssen, Laboratory Techniques in Biochemistry and Molecular Biology-Hybridization with Nucleic Acid Probes, part I chapter 2, Elsevier, N.Y. (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on a filter in a Southern or Northern blot is 50% formamide with 1 mg of heparin at 42° C., with the hybridization being carried out overnight. An example of highly stringent wash conditions is 0.15 M NaCl at 72° C. for about 15 minutes. Another example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An exemplary medium stringency wash for a duplex of, e.g., more than 100 nucleotides, is 1×SSC at 45° C. for 15 minutes. An example low stringency wash for a duplex of, e.g., more than 100 nucleotides, is 4×-6×SSC at 40° C. for 15 minutes. For short probes (e.g., about 10 to 50 nucleotides), stringent conditions typically involve salt concentrations of less than about 1.0 M sodium ions, typically about 0.01 to 1.0 M sodium ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

The following are examples of hybridization and wash conditions that may be used to identify nucleotide sequences that are substantially identical to reference nucleotide sequences of the present invention. A substantially identical nucleotide sequence preferably hybridizes to a reference nucleotide sequence in 7% sodium dodecyl sulfate (SDS), 0.5 M NaPO₄, 1 mM EDTA at 50° C. with washing in 2×SSC, 0.1% SDS at 50° C., more preferably in 7% sodium dodecyl sulfate (SDS), 0.5 M NaPO₄, 1 mM EDTA at 50° C. with washing in 1×SSC, 0.1% SDS at 50° C., still more preferably in 7% sodium dodecyl sulfate (SDS), 0.5 M NaPO₄, 1 mM EDTA at 50° C. with washing in 0.5×SSC, 0.1% SDS at 50° C., even more preferably in 7% sodium dodecyl sulfate (SDS), 0.5 M NaPO₄, 1 mM EDTA at 50° C. with washing in 0.1×SSC, 0.1% SDS at 50° C., and most preferably in 7% sodium dodecyl sulfate (SDS), 0.5 M NaPO₄, 1 mM EDTA at 50° C. with washing in 0.1×SSC, 0.1% SDS at 65° C.

A further indication that two nucleic acid sequences or proteins are substantially identical is that the that proteins encoded by the nucleic acids are substantially identical, share an overall three-dimensional structure, are biologically functional equivalents, or are immunologically cross-reactive with, or specifically bind to, each other. Nucleic acid molecules that do not hybridize to each other under stringent conditions are still substantially identical if the corresponding proteins are substantially identical. This may occur, for example, when two nucleotide sequences comprise conservatively substituted variants as permitted by the genetic code. This also includes degenerate codon substitutions wherein the third position of one or more selected (or all) codons is substituted with mixed-base and/or deoxyinosine residues (see Batzer et al., Nucleic Acids Res., 19:5081 (1991); Ohtsuka et al., J. Biol. Chem., 260:2605-2608 (1985); and Rossolini et al. Mol. Cell. Probes, 8:91-98 (1994)). However, both the polynucleotides and the polypeptides of the present invention may be conservatively substituted at one or more residues. Examples of conservative amino acid substitutions include the substitution of one non-polar (hydrophobic) residue such as isoleucine, valine, leucine or methionine for another; the substitution of one polar (hydrophilic) residue for another such as between arginine and lysine, between glutamine and asparagine, between glycine and serine; the substitution of one basic residue such as lysine, arginine or histidine for another; or the substitution of one acidic residue, such as aspartic acid or glutamic acid for another.

Nucleic acids of the invention also comprise nucleic acids complementary to SEQ ID NOs: 1 and 3 and subsequences and elongated sequences of SEQ ID NOs: 1 and 3 and complementary sequences thereof. Complementary sequences are two nucleotide sequences that comprise antiparallel nucleotide sequences capable of pairing with one another upon formation of hydrogen bonds between base pairs. Like other polynucleotides in accordance with the present invention, complementary sequences maybe substantially similar to one another as described previously. A particular example of a complementary nucleic acid segment is an antisense oligonucleotide.

A subsequence is a sequence of nucleic acids that comprises a part of a longer nucleic acid sequence. An exemplary subsequence is a probe or a primer. An elongated sequence is one in which nucleotides (or other analogous molecules) are added to a nucleic acid sequence. For example, a polymerase (e.g., a DNA polymerase) may add sequences at the 3′ terminus of the nucleic acid molecule. In addition, the nucleotide sequence may be combined with other DNA sequences, such as promoters, promoter regions, enhancers, polyadenylation signals, introns, additional restriction enzyme sites, multiple cloning sites, and other coding segments. Thus, the present invention also provides vectors comprising the disclosed nucleic acids, including vectors for recombinant expression, wherein a nucleic acid of the invention is operatively linked to a functional promoter. When operatively linked to a nucleic acid, a promoter is in functional combination with the nucleic acid such that the transcription of the nucleic acid is controlled and regulated by the promoter region. Vectors refer to nucleic acids capable of replication in a host cell, such as plasmids, cosmids, and viral vectors.

Polynucleotides of the present invention may be cloned, synthesized, altered, mutagenized, or combinations thereof. Standard recombinant DNA and molecular cloning techniques used to isolate nucleic acids are known in the art. Site-specific mutagenesis to create base pair changes, deletions, or small insertions is also known in the art (see e.g., Sambrook et al. (eds.) Molecular Cloning: A Laboratory Manual, 1989, Cold Spring Harbor Laboratory Press, Cold Spring Harbor, N.Y.; Silhavy et al., Experiments with Gene Fusions, 1984, Cold Spring Harbor Laboratory Press, Cold Spring Harbor, N.Y.; Glover & Hames, DNA Cloning: A Practical Approach, 2nd ed., 1995, IRL Press at Oxford University Press, Oxford/New York; Ausubel (ed.) Short Protocols in Molecular Biology, 3rd ed., 1995, Wiley, New York).

Isolated polypeptides of the invention may be purified and characterized using a variety of standard techniques that are known to the skilled artisan (see e.g., Schroder et al., The Peptides, 1965, Academic Press, New York; Bodanszky, Principles of Peptide Synthesis, 2nd rev. ed. 1993, Springer-Verlag, Berlin/New York; Ausubel (ed.), Short Protocols in Molecular Biology, 3rd ed., 1995, Wiley, New York).

The present invention also encompasses methods for detecting a nucleic acid molecule that encodes a WRKY protein. Such methods may be used to detect WRKY gene variants or altered gene expression. Sequences detected by methods of the invention may detected, subcloned, sequenced, and further evaluated by any measure well known in the art using any method usually applied to the detection of a specific DNA sequence. Thus, the nucleic acids of the present invention may be used to clone genes and genomic DNA comprising the disclosed sequences. Alternatively, the nucleic acids of the present invention may be used to clone genes and genomic DNA of related sequences. Levels of a WRKY nucleic acid molecule may be measured, for example, using an RT-PCR assay (see e.g., Chiang, J. Chromatogr. A., 806:209-218 (1998) and references cited therein).

The present invention also encompasses genetic assays using WRKY nucleic acids for quantitative trait loci (QTL) analysis and to screen for genetic variants, for example by allele-specific oligonucleotide (ASO) probe analysis (Conner et al., Proc. Natl. Acad. Sci. USA, 80(1):278-282 (1983)), oligonucleotide ligation assays (OLAs) (Nickerson et al., Proc. Natl. Acad. Sci. USA, 87(22):8923-8927 (1990)), single-strand conformation polymorphism (SSCP) analysis (Orita et al., Proc. Natl. Acad. Sci. USA, 86(8):2766-2770 (1989)), SSCP/heteroduplex analysis, enzyme mismatch cleavage, direct sequence analysis of amplified exons (Kestila et al., Mol. Cell, 1(4):575-582 (1998); Yuan et al., Hum. Mutat., 14(5):440-446 (1999)), allele-specific hybridization (Stoneking et al., Am. J. Hum. Genet., 48(2):370-382 (1991)), and restriction analysis of amplified genomic DNA containing the specific mutation. Automated methods may also be applied to large-scale characterization of single nucleotide polymorphisms (Wang et al., Am. J. Physiol., 1998, 274(4 Pt 2):H1132-1140 (1992); Brookes, Gene, 234(2):177-186 (1999)). Preferred detection methods are non-electrophoretic, including, for example, the TAQMAN™ allelic discrimination assay, PCR-OLA, molecular beacons, padlock probes, and well fluorescence (see Landegren et al., Genome Res., 8:769-776 (1998) and references cited therein).

The present invention also encompasses functional fragments of a WRKY polypeptide, for example, fragments that have the ability to alter a plant trait similar to that of SEQ ID NOs: 2 or 4. Functional polypeptide sequences that are longer than the disclosed sequences are also encompassed. For example, one or more amino acids may be added to the N-terminus or C-terminus of an antibody polypeptide. Such additional amino acids may be employed in a variety of applications, including but not limited to purification applications. Methods of preparing elongated proteins are known in the art.

The present invention also encompasses methods for detecting a WRKY polypeptide. Such methods can be used, for example, to determine levels of WRKY protein expression and correlate the level of expression with the presence or change in phenotype, trait, or level of expression in a different gene or gene product. In certain embodiments, the method involves an immunochemical reaction with an antibody that specifically recognizes a WRKY protein. Techniques for detecting such antibody-antigen conjugates or complexes are known in the art and include but are not limited to centrifugation, affinity chromatography and other immunochemical methods (see e.g., Ishikawa Ultrasensitive and Rapid Enzyme Immunoassay, 1999, Elsevier, Amsterdam/New York, United States of America; Law, Immunoassay: A Practical Guide, 1996, Taylor & Francis, London/Bristol, Pennsylvania, United States of America; Liddell et al., Antibody Technology, 1995, Bios Scientific Publishers, Oxford, United Kingdom; and references cited therein).

WRKY Expression Systems

An expression system refers to a host cell comprising a heterologous nucleic acid and the protein encoded by the heterologous nucleic acid. For example, a heterologous expression system may comprise a host cell transfected with a construct comprising a WRKY nucleic acid encoding a WRKY protein operatively linked to a promoter, or a cell line produced by introduction of WRKY nucleic acids into a host cell genome. The expression system may further comprise one or more additional heterologous nucleic acids relevant to WRKY function, such as targets of WRKY transcriptional activation or repression activity. These additional nucleic acids may be expressed as a single construct or multiple constructs.

A construct for expressing a WRKY protein may include a vector sequence and a WRKY nucleotide sequence, wherein the WRKY nucleotide sequence is operatively linked to a promoter sequence. A construct for recombinant WRKY expression may also comprise transcription termination signals and sequences required for proper translation of the nucleotide sequence. Preparation of an expression construct, including addition of translation and termination signal sequences, is known to one skilled in the art.

The promoter may be any polynucleotide sequence which shows transcriptional activity in the chosen plant cells, plant parts, or plants. The promoter may be native or analogous, or foreign or heterologous, to the plant host and/or to the DNA sequence of the invention. Where the promoter is native or endogenous to the plant host, it is intended that the promoter is found in the native plant into which the promoter is introduced. Where the promoter is foreign or heterologous to the DNA sequence of the invention, the promoter is not the native or naturally occurring promoter for the operably linked DNA sequence of the invention. The promoter may be inducible or constitutive. It may be naturally-occurring, may be composed of portions of various naturally-occurring promoters, or may be partially or totally synthetic. Guidance for the design of promoters is provided by studies of promoter structure, such as that of Harley et al., Nucleic Acids Res., 15:2343-61 (1987). Also, the location of the promoter relative to the transcription start may be optimized (see e.g., Roberts et al., Proc. Natl. Acad. Sci. USA, 76:760-4 (1979)). Many suitable promoters for use in plants are well known in the art.

For example, suitable constitutive promoters for use in plants include the promoters from plant viruses, such as the peanut chlorotic streak caulimovirus (PC1SV) promoter (U.S. Pat. No. 5,850,019); the 35S and 19S promoters from cauliflower mosaic virus (CaMV) (Odell et al., Nature, 313:810-812 (1985) and U.S. Pat. No. 5,352,605); the promoters of Chlorella virus methyltransferase genes (U.S. Pat. No. 5,563,328) and the full-length transcript promoter from figwort mosaic virus (FMV) (U.S. Pat. No. 5,378,619); the promoters from such genes as rice actin (McElroy et al., Plant Cell, 2:163-171 (1990)); ubiquitin (Binet et al., Plant Science, 79:87-94 (1991)), maize (Christensen et al., Plant Molec. Biol., 12: 619-632 (1989)), and arabidopsis (Norris et al., Plant Molec. Biol., 21:895-906 (1993); and Christensen et al., Plant Mol. Biol., 18:675-689 (1982)); pEMU (Last et al., Theor. Appl. Genet., 81:581-588 (1991)); MAS (Velten et al., EMBO J., 3:2723-2730 (1984)); maize H3 histone (Lepetit et al., Mol. Gen. Genet., 1992, 231:276-285 (1992); and Atanassova et al., Plant J., 2(3):291-300 (1992)); Brassica napus ALS3 (PCT International Publication No. WO 97/41228); and promoters of various Agrobacterium genes (e.g., U.S. Pat. Nos. 4,771,002; 5,102,796; 5,182,200; and 5,428,147).

Suitable inducible promoters for use in plants include the promoter from the ACE1 system which responds to copper (Mett et al., Proc. Natl. Acad. Sci. USA, 90:4567-4571 (1993)); the promoter of the maize 1n2 gene which responds to benzenesulfonamide herbicide safeners (Hershey et al., Mol. Gen. Genetics, 227:229-237 (1991); and Gatz et al., Mol. Gen. Genetics, 243:32-38 (1994)); and the promoter of the Tet repressor from Tn10 (Gatz et al., Mol. Gen. Genet., 227:229-237 (1991)). Another inducible promoter for use in plants is one that responds to an inducing agent to which plants do not normally respond. An exemplary inducible promoter of this type is the inducible promoter from a steroid hormone gene, the transcriptional activity of which is induced by a glucocorticosteroid hormone (Schena et al., Proc. Natl. Acad. Sci. USA, 88:10421 (1991)) or the recent application of a chimeric transcription activator, XVE, for use in an estrogen receptor-based inducible plant expression system activated by estradiol (Zuo et al., Plant J., 24:265-273 (2000)). Other inducible promoters for use in plants are described in EP 332104, PCT International Publication Nos. WO 93/21334 and WO 97/06269. Promoters composed of portions of other promoters and partially or totally synthetic promoters can also be used (see e.g., Ni et al., Plant J., 7:661-676 (1995) and PCT International Publication No. WO 95/14098 describing such promoters for use in plants).

Tissue-specific or tissue-preferential promoters useful for the expression of the genes of the invention in plants. Such promoters are disclosed in WO 93/07278. Other tissue specific promoters useful in the present invention include the cotton rubisco promoter disclosed in U.S. Pat. No. 6,040,504; the rice sucrose synthase promoter disclosed in U.S. Pat. No. 5,604,121; and the cestrum yellow leaf curling virus promoter disclosed in PCT International Publication No. WO 01/73087. Chemically inducible promoters useful for directing the expression of the novel dense and erect panicle gene in plants are disclosed in U.S. Pat. No. 5,614,395.

The promoter may include, or be modified to include, one or more enhancer elements to thereby provide for higher levels of transcription. Suitable enhancer elements for use in plants include the PC1SV enhancer element (U.S. Pat. No. 5,850,019), the CaMV 35S enhancer element (U.S. Pat. Nos. 5,106,739 and 5,164,316) and the FMV enhancer element (Maiti et al., Transgenic Res., 6:143-156 (1997)). See also PCT International Publication No. WO 96/23898.

Such constructs can contain a ‘signal sequence’ or ‘leader sequence’ to facilitate co-translational or post-translational transport of the peptide of interest to certain intracellular structures such as the chloroplast (or other plastid), endoplasmic reticulum, or Golgi apparatus, or to be secreted. For example, the construct can be engineered to contain a signal peptide to facilitate transfer of the peptide to the endoplasmic reticulum. A signal sequence is known or suspected to result in cotranslational or post-translational peptide transport across the cell membrane. In eukaryotes, this typically involves secretion into the Golgi apparatus, with some resulting glycosylation. A leader sequence refers to any sequence that, when translated, results in an amino acid sequence sufficient to trigger co-translational transport of the peptide chain to a sub-cellular organelle. Thus, this includes leader sequences targeting transport and/or glycosylation by passage into the endoplasmic reticulum, passage to vacuoles, plastids including chloroplasts, mitochondria, and the like. Plant expression cassettes may also contain an intron, such that mRNA processing of the intron is required for expression.

Such constructs can also contain 5′ and 3′ untranslated regions. A 3′ untranslated region is a polynucleotide located downstream of a coding sequence. Polyadenylation signal sequences and other sequences encoding regulatory signals capable of affecting the addition of polyadenylic acid tracts to the 3′ end of the mRNA precursor are 3′ untranslated regions. A 5′ untranslated region is a polynucleotide located upstream of a coding sequence.

The termination region may be native with the transcriptional initiation region, may be native with the sequence of the present invention, or may be derived from another source. Convenient termination regions are available from the Ti-plasmid of A. tumefaciens, such as the octopine synthase and nopaline synthase termination regions (see e.g., Guerineau et al., Mol. Gen. Genet., 262:141-144 (1991); Proudfoot, Cell, 64:671-674 (1991); Sanfacon et al., Genes Dev., 5:141-149 (1991); Mogen et al., Plant Cell, 2:1261-1272 (1990); Munroe et al., Gene, 91:151-158 (1990); Ballas et al., Nucleic Acids Res., 17:7891-7903 (1989); and Joshi et al., Nucleic Acid Res., 15:9627-9639 (1987)).

Where appropriate, the vector and WRKY sequences may be optimized for increased expression in the transformed host cell. That is, the sequences can be synthesized using host cell-preferred codons for improving expression, or may be synthesized using codons at a host-preferred codon usage frequency. Generally, the GC content of the polynucleotide will be increased (see e.g., Campbell et al., Plant Physiol., 92:1-11 (1990) for a discussion of host-preferred codon usage). Methods are known in the art for synthesizing host-preferred polynucleotides (see e.g., U.S. Pat. Nos. 6,320,100; 6,075,185; 5,380,831; and 5,436,391, U.S. Application Publication Nos. 20040005600 and 20010003849, and Murray et al., Nucleic Acids Res., 17:477-498 (1989).

In certain embodiments, polynucleotides of interest are targeted to the chloroplast for expression. In this manner, where the polynucleotide of interest is not directly inserted into the chloroplast, the expression cassette may additionally contain a polynucleotide encoding a transit peptide to direct the nucleotide of interest to the chloroplasts. Such transit peptides are known in the art (see e.g., Von Heijne et al., Plant Mol. Biol. Rep., 9:104-126 (1991); Clark et al., J. Biol. Chem., 264:17544-17550 (1989); Della-Cioppa et al., Plant Physiol., 84:965-968 (1987); Romer et al., Biochem. Biophys. Res. Commun., 196:1414-1421 (1993); and Shah et al., Science, 233:478-481 (1986)). The polynucleotides of interest to be targeted to the chloroplast may be optimized for expression in the chloroplast to account for differences in codon usage between the plant nucleus and this organelle. In this manner, the polynucleotides of interest may be synthesized using chloroplast-preferred codons (see e.g., U.S. Pat. No. 5,380,831).

A plant expression cassette (i.e., a WRKY open reading frame operatively linked to a promoter) can be inserted into a plant transformation vector, which allows for the transformation of DNA into a cell. Such a molecule may consist of one or more expression cassettes, and may be organized into more than one vector DNA molecule. For example, binary vectors are plant transformation vectors that utilize two non-contiguous DNA vectors to encode all requisite cis- and trans-acting functions for transformation of plant cells (Hellens et al., Trends in Plant Science, 5:446-451 (2000)).

A plant transformation vector comprises one or more DNA vectors for achieving plant transformation. For example, it is a common practice in the art to utilize plant transformation vectors that comprise more than one contiguous DNA segment. These vectors are often referred to in the art as binary vectors. Binary vectors as well as vectors with helper plasmids are most often used for Agrobacterium-mediated transformation, where the size and complexity of DNA segments needed to achieve efficient transformation is quite large, and it is advantageous to separate functions onto separate DNA molecules. Binary vectors typically contain a plasmid vector that contains the cis-acting sequences required for T-DNA transfer (such as left border and right border), a selectable marker that is engineered to be capable of expression in a plant cell, and a polynucleotide of interest (i.e., a polynucleotide engineered to be capable of expression in a plant cell for which generation of transgenic plants is desired).

For certain target species, different antibiotic or herbicide selectable markers may be preferred. Selection markers used routinely in transformation include the nptII gene, which confers resistance to kanamycin and related antibiotics (Messing & Vierra, Gene, 19:259-268 (1982); and Bevan et al., Nature, 304:184-187 (1983)), the bar gene, which confers resistance to the herbicide phosphinothricin (White et al., Nucl. Acids Res., 18: 1062 (1990), and Spencer et al., Theor. Appl. Genet., 79: 625-631 (1990)), the hph gene, which confers resistance to the antibiotic hygromycin (Blochinger & Diggelmann, Mol. Cell. Biol., 4:2929-2931 (1984)), the dhfr gene, which confers resistance to methotrexate (Bourouis et al., EMBO J., 2(7):1099-1104 (1983)), the EPSPS gene, which confers resistance to glyphosate (U.S. Pat. Nos. 4,940,935 and 5,188,642), and the mannose-6-phosphate isomerase gene, which provides the ability to metabolize mannose (U.S. Pat. Nos. 5,767,378 and 5,994,629).

Also present on this plasmid vector are sequences required for bacterial replication. The cis-acting sequences are arranged in a fashion to allow efficient transfer into plant cells and expression therein. For example, the selectable marker sequence and the sequence of interest are located between the left and right borders. Often a second plasmid vector contains the trans-acting factors that mediate T-DNA transfer from Agrobacterium to plant cells. This plasmid often contains the virulence functions (Vir genes) that allow infection of plant cells by Agrobacterium, and transfer of DNA by cleavage at border sequences and vir-mediated DNA transfer, as in understood in the art (Hellens et al., 2000). Several types of Agrobacterium strains (e.g., LBA4404, GV3101, EHA101, EHA105, etc.) can be used for plant transformation. The second plasmid vector is not necessary for introduction of polynucleotides into plants by other methods such as, e.g., microprojection, microinjection, electroporation, and polyethylene glycol.

In another embodiment, a nucleotide sequence of the present invention is directly transformed into a plastid genome. A major advantage of plastid transformation is that plastids are generally capable of expressing bacterial genes without substantial modification, and plastids are capable of expressing multiple open reading frames under control of a single promoter. Plastid transformation technology is extensively described in U.S. Pat. Nos. 5,451,513, 5,545,817 and 5,545,818, in PCT International Application Publication WO 95/16783, and in McBride et al., Proc. Natl. Acad. Sci. USA, 91:7301-7305 (1994). The basic technique for chloroplast transformation involves introducing regions of cloned plastid DNA flanking a selectable marker together with the gene of interest into a suitable target tissue, e.g., using biolistics or protoplast transformation (e.g., calcium chloride or PEG mediated transformation). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rpsl2 genes conferring resistance to spectinomycin and/or streptomycin are utilized as selectable markers for transformation (Svab et al., Proc. Natl. Acad. Sci. USA, 87:8526-8530 (1990); Staub et al., Plant Cell, 4:39-45 (1992)). This results in stable homoplasmic transformants at a frequency of approximately one per 100 bombardments of target leaves. The presence of cloning sites between these markers allows creation of a plastid targeting vector for introduction of foreign genes (Staub et al., EMBO J., 12:601-606 (1993)). Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase (Svab et al., Proc. Natl. Acad. Sci. USA, 90:913-917 (1993)). Previously, this marker had been used successfully for high-frequency transformation of the plastid genome of the green alga Chlamydomonas reinhardtii (Goldschmidt-Clermont, Nucl. Acids Res., 19:4083-4089 (1991)). Other selectable markers useful for plastid transformation are known in the art. Typically, approximately 15-20 cell division cycles following transformation are required to reach a homoplastidic state. Plastid expression, in which genes are inserted by homologous recombination into all of the several thousand copies of the circular plastid genome present in each plant cell, takes advantage of the enormous copy number advantage over nuclear-expressed genes to permit expression levels that can readily exceed 10% of the total soluble plant protein. In a preferred embodiment, a nucleotide sequence of the present invention is inserted into a plastid-targeting vector and transformed into the plastid genome of a desired plant host. Plants homoplastic for plastid genomes containing a nucleotide sequence of the present invention are obtained, and are preferentially capable of high expression of the nucleotide sequence.

Host Cells

Host cells are cells into which a heterologous nucleic acid molecule of the invention may be introduced. Representative eukaryotic host cells include yeast and plant cells, as well as prokaryotic hosts such as E. coli and B. subtilis. Preferred host cells for functional assays substantially or completely lack endogenous expression of a WRKY protein.

A host cell strain may be chosen which modulates the expression of the recombinant sequence, or modifies and processes the gene product in a specific manner. For example, different host cells have characteristic and specific mechanisms for the translational and post-translational processing and modification (e.g., glycosylation, phosphorylation of proteins). Appropriate cell lines or host cells may be chosen to ensure the desired modification and processing of the foreign protein expressed. For example, expression in a bacterial system may be used to produce a non-glycosylated core protein product, and expression in yeast will produce a glycosylated product.

The present invention further encompasses recombinant expression of a WRKY protein in a stable cell line. Methods for generating a stable cell line following transformation of a heterologous construct into a host cell are known in the art (see e.g., Joyner, Gene Targeting: A Practical Approach, 1993, Oxford University Press, Oxford/New York). Thus, transformed cells, tissues, and plants are understood to encompass not only the end product of a transformation process, but also transgenic progeny or propagated forms thereof.

WRKY Knockout Plants

The present invention also provides WRKY knockout plants comprising a disruption of a WRKY locus. A disrupted gene may result in expression of an altered level of full-length WRKY protein or expression of a mutated variant WRKY protein. Plants with complete or partial functional inactivation of the WRKY gene may be generated, e.g., by expressing an amorphic (i.e., null mutation) or hypomorphic WRKY allele in the plant.

A knockout plant in accordance with the present invention may also be prepared using anti-sense, double-stranded RNA, or ribozyme WRKY constructs, driven by a universal or tissue-specific promoter to reduce levels of WRKY gene expression in somatic cells, thus achieving a “knock-down” phenotype. The present invention also provides the generation of plants with conditional or inducible inactivation of WRKY.

The present invention also encompasses transgenic plants with specific “knocked-in” modifications in the disclosed WRKY gene. In certain embodiments, a “knocked-in” transgenic plant expresses an antimorphic (i.e., dominant negative) allele. In other embodiments, a “knocked-in” transgenic plant expresses a hypermorphic (i.e., a gain of function) allele.

WRKY knockout plants may be prepared in monocot or dicot plants, such as maize, wheat, barley, rye, sweet potato, bean, pea, chicory, lettuce, cabbage, cauliflower, broccoli, turnip, radish, spinach, asparagus, onion, garlic, pepper, celery, squash, pumpkin, hemp, zucchini, apple, pear, quince, melon, plum, cherry, peach, nectarine, apricot, strawberry, grape, raspberry, blackberry, pineapple, avocado, papaya, mango, banana, soybean, tomato, sorghum, sugarcane, sugar beet, sunflower, rapeseed, clover, tobacco, carrot, cotton, alfalfa, rice, potato, eggplant, cucumber, Arabidopsis, and woody plants such as coniferous and deciduous trees. Rice, wheat, barley, oat, soybean and rye are particularly contemplated. As used herein, a plant refers to a whole plant, a plant organ (e.g., root, stem, leaf, flower bud, or embryo), a seed, a plant cell, a propagule, an embryo, other plant parts (e.g., protoplasts, pollen, pollen tubes, ovules, embryo sacs, zygotes) and progeny of the same. Plant cells can be differentiated or undifferentiated (e.g., callus, suspension culture cells, protoplasts, leaf cells, root cells, phloem cells, pollen).

For preparation of a WRKY knockout plant, introduction of a polynucleotide into plant cells is accomplished by one of several techniques known in the art, including but not limited to electroporation or chemical transformation (see e.g., Ausubel, ed. (1994) Current Protocols in Molecular Biology, John Wiley and Sons, Inc., Indianapolis, Ind.). Markers conferring resistance to toxic substances are useful in identifying transformed cells (having taken up and expressed the test polynucleotide sequence) from non-transformed cells (those not containing or not expressing the test polynucleotide sequence). In one aspect of the invention, genes are useful as a marker to assess introduction of DNA into plant cells. Transgenic plants, transformed plants, or stably transformed plants, or cells, tissues or seed of any of the foregoing, refer to plants that have incorporated or integrated exogenous polynucleotides into the plant cell. Stable transformation refers to introduction of a polynucleotide construct into a plant such that it integrates into the genome of the plant and is capable of being inherited by progeny thereof.

In general, plant transformation methods involve transferring heterologous DNA into target plant cells (e.g., immature or mature embryos, suspension cultures, undifferentiated callus, protoplasts, etc.), followed by applying a maximum threshold level of appropriate selection (depending on the selectable marker gene) to recover the transformed plant cells from a group of untransformed cell mass. Explants are typically transferred to a fresh supply of the same medium and cultured routinely. Subsequently, the transformed cells are differentiated into shoots after placing on regeneration medium supplemented with a maximum threshold level of selecting agent (i.e., temperature and/or herbicide). The shoots are then transferred to a selective rooting medium for recovering rooted shoot or plantlet. The transgenic plantlet then grow into mature plant and produce fertile seeds (see e.g., Hiei et al., Plant J., 6:271-282 (1994); and Ishida et al., Nat. Biotechnol., 14:745-750 (1996)). A general description of the techniques and methods for generating transgenic plants are found in Ayres et al., CRC Crit. Rev. Plant Sci., 13:219-239 (1994); and Bommineni et al., Maydica, 42:107-120 (1997). Since the transformed material contains many cells, both transformed and non-transformed cells are present in any piece of subjected target callus or tissue or group of cells. The ability to kill non-transformed cells and allow transformed cells to proliferate results in transformed plant cultures. Often, the ability to remove non-transformed cells is a limitation to rapid recovery of transformed plant cells and successful generation of transgenic plants. Molecular and biochemical methods can then be used for confirming the presence of the integrated nucleotide(s) of interest in the genome of transgenic plant.

Generation of transgenic plants may be performed by one of several methods, including but not limited to introduction of heterologous DNA by Agrobacterium into plant cells (Agrobacterium-mediated transformation), bombardment of plant cells with heterologous foreign DNA adhered to particles, and various other non-particle direct-mediated methods to transfer DNA (see e.g., Hiei et al., Plant J., 6:271-282 (1994); Ishida et al., Nat. Biotechnol., 14:745-750 (1996); Ayres et al., CRC Crit. Rev. Plant Sci., 13:219-239 (1994); and Bommineni et al., Maydica, 1997, 42:107-120 (1997)).

There are three common methods to transform plant cells with Agrobacterium. The first method is co-cultivation of Agrobacterium with cultured isolated protoplasts. This method requires an established culture system that allows culturing protoplasts and plant regeneration from cultured protoplasts. The second method is transformation of cells or tissues with Agrobacterium. This method requires (a) that the plant cells or tissues can be transformed by Agrobacterium and (b) that the transformed cells or tissues can be induced to regenerate into whole plants. The third method is transformation of seeds, apices or meristems with Agrobacterium. This method requires micropropagation.

The efficiency of transformation by Agrobacterium may be enhanced by using a number of methods known in the art. For example, the inclusion of a natural wound response molecule such as acetosyringone (AS) to the Agrobacterium culture has been shown to enhance transformation efficiency with Agrobacterium tumefaciens (Shahla et al., Plant Molec. Biol, 8:291-298 (1987)). Alternatively, transformation efficiency may be enhanced by wounding the target tissue to be transformed. Wounding of plant tissue may be achieved, for example, by punching, maceration, bombardment with microprojectiles (see e.g., Bidney et al., Plant Molec. Biol., 18:301-313 (1992).

In one embodiment, the plant cells are transfected with vectors via particle bombardment (i.e., with a gene gun). Particle mediated gene transfer methods are known in the art, are commercially available, and include, but are not limited to, the gas driven gene delivery instrument described in U.S. Pat. No. 5,584,807. This method involves coating the polynucleotide sequence of interest onto heavy metal particles, and accelerating the coated particles under the pressure of compressed gas for delivery to the target tissue.

Other particle bombardment methods are also available for the introduction of heterologous polynucleotide sequences into plant cells. Generally, these methods involve depositing the polynucleotide sequence of interest upon the surface of small, dense particles of a material such as gold, platinum, or tungsten. The coated particles are themselves then coated onto either a rigid surface, such as a metal plate, or onto a carrier sheet made of a fragile material such as mylar. The coated sheet is then accelerated toward the target biological tissue. The use of the flat sheet generates a uniform spread of accelerated particles that maximizes the number of cells receiving particles under uniform conditions, resulting in the introduction of the polynucleotide sample into the target tissue.

Specific initiation signals may also be used to achieve more efficient translation of sequences encoding the polypeptide of interest. Such signals include the ATG initiation codon and adjacent sequences. In cases where sequences encoding the polypeptide of interest, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers that are appropriate for the particular cell system that is used, such as those described in the literature (Scharf et al., Results Probl. Cell Differ., 20:125 (1994)).

The cells that have been transformed may be grown into plants in accordance with conventional ways (see e.g., McCormick et al., Plant Cell Rep., 5:81-84 (1986)). These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as transgenic seed) having a polynucleotide of the invention, for example, an expression cassette of the invention, stably incorporated into their genome.

Transgenic plants of the invention can be homozygous for the added polynucleotides; i.e., a transgenic plant that contains two added sequences, one sequence at the same locus on each chromosome of a chromosome pair. A homozygous transgenic plant can be obtained by sexually mating (selfing) an independent segregant transgenic plant that contains the added sequences according to the invention, germinating some of the seed produced and analyzing the resulting plants produced for enhanced enzyme activity (i.e., herbicide resistance) and/or increased plant yield relative to a control (native, non-transgenic) or an independent segregant transgenic plant.

It is to be understood that two different transgenic plants can also be mated to produce offspring that contain two independently segregating added, exogenous polynucleotides. Selfing of appropriate progeny can produce plants that are homozygous for all added exogenous polynucleotides that encode a polypeptide of the present invention. Back-crossing to a parental plant and outcrossing with a non-transgenic plant are also contemplated.

Following introduction of DNA into plant cells, the transformation or integration of the polynucleotide into the plant genome is confirmed by various methods such as analysis of polynucleotides, polypeptides and metabolites associated with the integrated sequence.

WRKY Inhibitors

The present invention further discloses assays to identify WRKY binding partners and WRKY inhibitors. WRKY antagonists/inhibitors are agents that alter the function of a WRKY protein e.g., by altering chemical and biological activities or properties. Methods of identifying inhibitors involve assaying a reduced level or quality of WRKY function in the presence of one or more agents. Exemplary WRKY inhibitors include small molecules as well as biological inhibitors as described herein below.

As used herein, the term “agent” refers to any substance that potentially interacts with a WRKY nucleic acid or protein, including any of synthetic, recombinant, or natural origin. An agent suspected to interact with a protein may be evaluated for such an interaction using the methods disclosed herein.

Exemplary agents include but are not limited to peptides, proteins, nucleic acids, small molecules (e.g., chemical compounds), antibodies or fragments thereof, nucleic acid-protein fusions, any other affinity agent, and combinations thereof. An agent to be tested may be a purified molecule, a homogenous sample, or a mixture of molecules or compounds.

A small molecule refers to a compound, for example an organic compound, with a molecular weight of less than about 1,000 daltons, more preferably less than about 750 daltons, still more preferably less than about 600 daltons, and still more preferably less than about 500 daltons. A small molecule also preferably has a computed log octanol-water partition coefficient in the range of about −4 to about +14, more preferably in the range of about −2 to about +7.5.

Exemplary nucleic acids that may be used to disrupt WRKY function include antisense RNA and small interfering RNAs (siRNAs) (see e.g., U.S. Application Publication No. 20060095987. These inhibitory molecules may be prepared based upon the WRKY gene sequence and known features of inhibitory nucleic acids (see e.g., Van der Krol et al., Plant Cell, 2:291-299 (1990); Napoli et al., Plant Cell, 2:279-289 (1990); English et al., Plant Cell, 8:179-188 (1996); and Waterhouse et al., Nature Rev. Genet., 2003, 4:29-38 (2003).

Agents may be obtained or prepared as a library or collection of molecules. A library may contain a few or a large number of different molecules, varying from about ten molecules to several billion molecules or more. A molecule may comprise a naturally occurring molecule, a recombinant molecule, or a synthetic molecule. A plurality of agents in a library may be assayed simultaneously. Optionally, agents derived from different libraries may be pooled for simultaneous evaluation.

Representative libraries include but are not limited to a peptide library (U.S. Pat. Nos. 6,156,511, 6,107,059, 5,922,545, and 5,223,409), an oligomer library (U.S. Pat. Nos. 5,650,489 and 5,858,670), an aptamer library (U.S. Pat. Nos. 7,338,762; 7,329,742; 6,949,379; 6,180,348; and 5,756,291), a small molecule library (U.S. Pat. Nos. 6,168,912 and 5,738,996), a library of antibodies or antibody fragments (U.S. Pat. Nos. 6,174,708, 6,057,098, 5,922,254, 5,840,479, 5,780,225, 5,702,892, and 5,667,988), a library of nucleic acid-protein fusions (U.S. Pat. No. 6,214,553), and a library of any other affinity agent that may potentially bind to a WRKY protein.

A library may comprise a random collection of molecules. Alternatively, a library may comprise a collection of molecules having a bias for a particular sequence, structure, or conformation, for example, as for inhibitory nucleic acids (see e.g., U.S. Pat. Nos. 5,264,563 and 5,824,483). Methods for preparing libraries containing diverse populations of various types of molecules are known in the art, for example as described in U.S. patents cited herein above. Numerous libraries are also commercially available.

A control level or quality of WRKY activity refers to a level or quality of wild type WRKY activity, for example, when using a recombinant expression system comprising expression of SEQ ID NO: 2. When evaluating the inhibiting capacity of an agent, a control level or quality of WRKY activity comprises a level or quality of activity in the absence of the agent. A control level may also be established by a phenotype or other measureable trait.

Methods of identifying WRKY inhibitors also require that the inhibiting capacity of an agent be assayed. Assaying the inhibiting capacity of an agent may comprise determining a level of WRKY gene expression; determining DNA binding activity of a recombinantly expressed WRKY protein; determining an active conformation of a WRKY protein; or determining a change in a trait in response to binding of a WRKY inhibitor (e.g., drought/osmotic stress tolerance, salt stress tolerance and cold/freezing stress tolerance). In particular embodiments, a method of identifying a WRKY inhibitor may comprise (a) providing a cell, plant, or plant part expressing a WRKY protein; (b) contacting the cell, plant, or plant part with an agent; (c) examining the cell, plant, or plant part for a change in a trait as compared to a control; and (d) selecting an agent that induces a change in the trait as compared to a control. Any of the agents so identified in the disclosed inhibitory or binding assays (see hereinafter) may be subsequently applied to a cell, plant or plant part as desired to effectuate a change in that cell, plant or plant part. For example, disruption of a WRKY gene (e.g., SEQ ID NO: 1) or inhibition of a WRKY polynucleotide or polypeptide (e.g., SEQ ID NO: 2) would likely alter one or more plant traits in a non-desirable fashion (e.g., decrease stress tolerance).

The present invention also encompasses a rapid and high throughput screening method that relies on the methods described herein. This screening method comprises separately contacting a WRKY protein with a plurality of agents. In such a screening method the plurality of agents may comprise more than about 10⁴ samples, or more than about 10⁵ samples, or more than about 10⁶ samples.

The in vitro and cellular assays of the invention may comprise soluble assays, or may further comprise a solid phase substrate for immobilizing one or more components of the assay. For example, a WRKY protein, or a cell expressing a WRKY protein, may be bound directly to a solid state component via a covalent or non-covalent linkage. Optionally, the binding may include a linker molecule or tag that mediates indirect binding of a WRKY protein to a substrate.

WRKY Binding Assays

The present invention also encompasses methods of identifying of a WRKY inhibitor by determining specific binding of a substance (e.g., an agent described previously) to a WRKY protein. For example, a method of identifying a WRKY binding partner may comprise: (a) providing a WRKY protein of any one of SEQ ID NOs: 2 and 4; (b) contacting the WRKY protein with one or more agents under conditions sufficient for binding; (c) assaying binding of the agent to the isolated WRKY protein; and (d) selecting an agent that demonstrates specific binding to the WRKY protein. Specific binding may also encompass a quality or state of mutual action such that binding of an agent to a WRKY protein is inhibitory.

Specific binding refers to a binding reaction which is determinative of the presence of the protein in a heterogeneous population of proteins and other biological materials. The binding of an agent to a WRKY protein may be considered specific if the binding affinity is about 1×10⁴M¹ to about 1×10⁶M⁻¹ or greater. Specific binding also refers to saturable binding. To demonstrate saturable binding of an agent to a WRKY protein, Scatchard analysis may be carried out as described, for example, by Mak et al., J. Biol. Chem., 264:21613-21618 (1989).

Several techniques may be used to detect interactions between a WRKY protein and an agent without employing a known competitive inhibitor. Representative methods include, but are not limited to, Fluorescence Correlation Spectroscopy, Surface-Enhanced Laser Desorption/Ionization Time-Of-Flight Spectroscopy, and BIACORE® technology, each technique described herein below. These methods are amenable to automated, high-throughput screening.

Fluorescence Correlation Spectroscopy (FCS) measures the average diffusion rate of a fluorescent molecule within a small sample volume. The sample size may be as low as 10³ fluorescent molecules and the sample volume as low as the cytoplasm of a single bacterium. The diffusion rate is a function of the mass of the molecule and decreases as the mass increases. FCS may therefore be applied to protein-ligand interaction analysis by measuring the change in mass and therefore in diffusion rate of a molecule upon binding. In a typical experiment, the target to be analyzed (e.g., a WRKY protein) is expressed as a recombinant protein with a sequence tag, such as a poly-histidine sequence, inserted at the N-terminus or C-terminus. The expression is mediated in a host cell, such as E. coli, yeast, Xenopus oocytes, or mammalian cells. The protein is purified using chromatographic methods. For example, the poly-histidine tag may be used to bind the expressed protein to a metal chelate column such as Ni²⁺ chelated on iminodiacetic acid agarose. The protein is then labeled with a fluorescent tag such as carboxytetramethylrhodamine or BODIPY™ reagent (available from Molecular Probes of Eugene, Oreg.). The protein is then exposed in solution to the potential ligand, and its diffusion rate is determined by FCS using instrumentation available from Carl Zeiss, Inc. (Thornwood of New York, N.Y.). Ligand binding is determined by changes in the diffusion rate of the protein.

Surface-Enhanced Laser Desorption/Ionization (SELDI) was developed by Hutchens & Yip, Rapid Commun. Mass Spectrom., 1993, 7:576-580. When coupled to a time-of-flight mass spectrometer (TOF), SELDI provides a technique to rapidly analyze molecules retained on a chip. It may be applied to ligand-protein interaction analysis by covalently binding the target protein, or portion thereof, on the chip and analyzing by mass spectrometry the small molecules that bind to this protein (Worrall et al., Anal Chem., 1998, 70(4):750-756 (1998)). In a typical experiment, a target protein (e.g., a WRKY protein) is recombinantly expressed and purified. The target protein is bound to a SELDI chip either by utilizing a poly-histidine tag or by other interaction such as ion exchange or hydrophobic interaction. A chip thus prepared is then exposed to the potential ligand via, for example, a delivery system able to pipet the ligands in a sequential manner (autosampler). The chip is then washed in solutions of increasing stringency, for example a series of washes with buffer solutions containing an increasing ionic strength. After each wash, the bound material is analyzed by submitting the chip to SELDI-TOF. Ligands that specifically bind a target protein are identified by the stringency of the wash needed to elute them.

BIACORE® relies on changes in the refractive index at the surface layer upon binding of a ligand to a target protein (e.g., a WRKY protein) immobilized on the layer. In this system, a collection of small ligands is injected sequentially in a 2-5 microliter cell, wherein the target protein is immobilized within the cell. Binding is detected by surface plasmon resonance (SPR) by recording laser light refracting from the surface. In general, the refractive index change for a given change of mass concentration at the surface layer is practically the same for all proteins and peptides, allowing a single method to be applicable for any protein. In a typical experiment, a target protein is recombinantly expressed, purified, and bound to a BIACORE® chip. Binding may be facilitated by utilizing a poly-histidine tag or by other interaction such as ion exchange or hydrophobic interaction. A chip thus prepared is then exposed to one or more potential ligands via the delivery system incorporated in the instruments sold by Biacore (Uppsala, Sweden) to pipet the ligands in a sequential manner (autosampler). The SPR signal on the chip is recorded and changes in the refractive index indicate an interaction between the immobilized target and the ligand. Analysis of the signal kinetics of on rate and off rate allows the discrimination between non-specific and specific interaction (see also Homola et al., Sensors and Actuators, 54:3-15 (1999) and references therein).

Conformational Assays

The present invention also encompasses methods of identifying WRKY binding partners and inhibitors that rely on a conformational change of a WRKY protein when bound by or otherwise interacting with a substance (e.g., an agent described previously). For example, application of circular dichroism to solutions of macromolecules reveals the conformational states of these macromolecules. The technique may distinguish random coil, alpha helix, and beta chain conformational states.

To identify inhibitors of a WRKY protein, circular dichroism analysis may be performed using a recombinantly expressed WRKY protein. A WRKY protein is purified, for example by ion exchange and size exclusion chromatography, and mixed with an agent. The mixture is subjected to circular dichroism. The conformation of a WRKY protein in the presence of an agent is compared to a conformation of a WRKY protein in the absence of the agent. A change in conformational state of a WRKY protein in the presence of an agent identifies a WRKY binding partner or inhibitor. Representative methods are described in U.S. Pat. Nos. 5,776,859 and 5,780,242. Antagonistic activity of the inhibitor may be assessed using functional assays, such as assaying for altered stress tolereance as described herein.

In accordance with the disclosed methods, cells expressing WRKY may be provided in the form of a kit useful for performing an assay of WRKY function. For example, a kit for detecting a WRKY may include cells transfected with DNA encoding a full-length WRKY protein and a medium for growing the cells.

Assays of WRKY activity that employ transiently transfected cells may include a marker that distinguishes transfected cells from non-transfected cells. A marker may be encoded by or otherwise associated with a construct for WRKY expression, such that cells are simultaneously transfected with a nucleic acid molecule encoding WRKY and the marker. Representative detectable molecules that are useful as markers include but are not limited to a heterologous nucleic acid, a protein encoded by a transfected construct (e.g., an enzyme or a fluorescent protein), a binding protein, and an antigen.

Assays employing cells expressing recombinant WRKY or plants expressing WRKY may additionally employ control cells or plants that are substantially devoid of native WRKY and, optionally, proteins substantially similar to a WRKY protein. When using transiently transfected cells, a control cell may comprise, for example, an untransfected host cell. When using a stable cell line expressing a WRKY protein, a control cell may comprise, for example, a parent cell line used to derive the WRKY-expressing cell line.

Anti-WRKY Antibodies

In another aspect of the invention, a method is provided for producing an antibody that specifically binds a WRKY protein. According to the method, a full-length recombinant WRKY protein is formulated so that it may be used as an effective immunogen, and used to immunize an animal so as to generate an immune response in the animal. The immune response is characterized by the production of antibodies that may be collected from the blood serum of the animal.

An antibody is an immunoglobulin protein, or antibody fragments that comprise an antigen binding site (e.g., Fab, modified Fab, Fab′, F(ab′)₂ or Fv fragments, or a protein having at least one immunoglobulin light chain variable region or at least one immunoglobulin heavy chain region). Antibodies of the invention include diabodies, tetrameric antibodies, single chain antibodies, tretravalent antibodies, multispecific antibodies (e.g., bispecific antibodies), and domain-specific antibodies that recognize a particular epitope. Cell lines that produce anti-WRKY antibodies are also encompassed by the invention.

Specific binding of an antibody to a WRKY protein refers to preferential binding to a WRKY protein in a heterogeneous sample comprising multiple different antigens. Substantially lacking binding describes binding of an antibody to a control protein or sample, i.e., a level of binding characterized as non-specific or background binding. The binding of an antibody to an antigen is specific if the binding affinity is at least about 10⁻⁷ M or higher, such as at least about 10⁻⁸ M or higher, including at least about 10⁻⁹ M or higher, at least about 10⁻¹¹ M or higher, or at least about 10⁻¹² M or higher.

WRKY antibodies prepared as disclosed herein may be used in methods known in the art relating to the expression and activity of WRKY proteins, e.g., for cloning of nucleic acids encoding a WRKY protein, immunopurification of a WRKY protein, and detecting a WRKY protein in a plant sample, and measuring levels of a WRKY protein in plant samples. To perform such methods, an antibody of the present invention may further comprise a detectable label, including but not limited to a radioactive label, a fluorescent label, an epitope label, and a label that may be detected in vivo. Methods for selection of a label suitable for a particular detection technique, and methods for conjugating to or otherwise associating a detectable label with an antibody are known to one skilled in the art.

### EXAMPLES

The invention is now described with reference to the following Examples. These Examples are provided for the purpose of illustration only, and the invention is not limited to these Examples, but rather encompasses all variations which are evident as a result of the teachings provided herein.

### Example 1

### Identification of WRKY Genes from Wheat

The WRKY proteins contain a conserved WRKY motif (WRKYGQK) and a novel zinc finger-like motif (C₂—H₂ or C₂—H—C motif). By using consensus sequences of the two motifs for a basic local alignment search (BLAST) against 494,195 expressed sequence tags (ESTs) from wheat, many ESTs were identified. After sequence assembly and subsequent removal of those sequences without WRKY motif, putative WRKY unigenes were identified. The full-length coding regions of TaWRKY-2 (SEQ ID NO: 1) and TaWRKY-19 (SEQ ID NO: 3) were obtained by using RT-PCR and RACE. The open reading frames for TaWRKY2 and TaWRKY19 were both 1407 bp in length, and encoded proteins 468 amino acids in length (i.e., TaWRKY-2 (SEQ ID NO: 2) and TaWRKY-19 (SEQ ID NO: 4)). Except for the WRKY domains, TaWRKY-2 and TaWRKY-19 shared little identity.

### Example 2

### Expression of TaWRKY Genes in Response to Various Abiotic Stresses The seeds of wheat (Triticicum aestivum L. cultivar Xifeng 20) were germinated on moistened gauze at 25° C. after being immersed in water for 2 days at 37° C. and then grown hydroponically on gauze immersed in water in petri dishes at 25° C. under continuous light (approximately 2500 lux). Fifteen day old seedlings were then subjected to drought, 250 mM NaCl, 4° C., wounding and 100 μM ABA.

In the drought stress experiments, seedlings were transferred onto filter paper and dried for 0, 1, 3, 6, 12, and 24 hours at 25° C. and 20% relative humidity. For NaCl and ABA treatment, the seedling roots were immersed in water containing 200 mM NaCl and 100 μM ABA, respectively, for 0, 1, 3, 6, 12, and 24 hours. For 4° C. treatment, the seedlings were grown hydroponically at 4° C. for 0, 1, 3, 6, 12, and 24 hours. After treatment, leaves were corrected and frozen immediately in liquid nitrogen and stored at −70° C. for total RNA preparation. Roots, stems and leaves from 20-day-old seedlings grown under normal growth conditions were also harvested for further analysis.

Frozen tissues were ground into a fine powder in liquid nitrogen with a mortar and pistil for total RNA isolation. Total RNA was isolated by using guanidine thiocyanate method and purified by phenol-chloroform extraction. The first-strand cDNA, a template to amplification in RT-PCR, was synthesized following the instructions of a first-strand cDNA synthesis kit (Promega). The specific primers were designed matching the TaWRKY gene sequences. The total volume of the PCR reaction mixture was 15 including 1 μl of the first-strand cDNA, 0.2 μM of each primer, 1×PCR buffer (containing 1.5 mM Mg²⁺), 0.2 mM dNTP and 1 unit of Taq DNA polymerase (Takara). Amplification was carried out with the program of 94° C. for 3 minutes for denaturation; 30 cycles of 94° C. for 1 minute, 58° C. (TaWRKY2), 52° C. (TaWRKY19), or 54° C. (actin) for 1 minute, and 72° C. for 1 minute; and 72° C. for 10 minutes for final extension. A wheat actin gene was amplified as a control. The amplification products were separated on a 1% w/v agarose gel, stained with ethidium bromide and evaluated by using a Gel Doc GS670 gel imaging system (Bio-Rad, Hercules, Calif., USA).

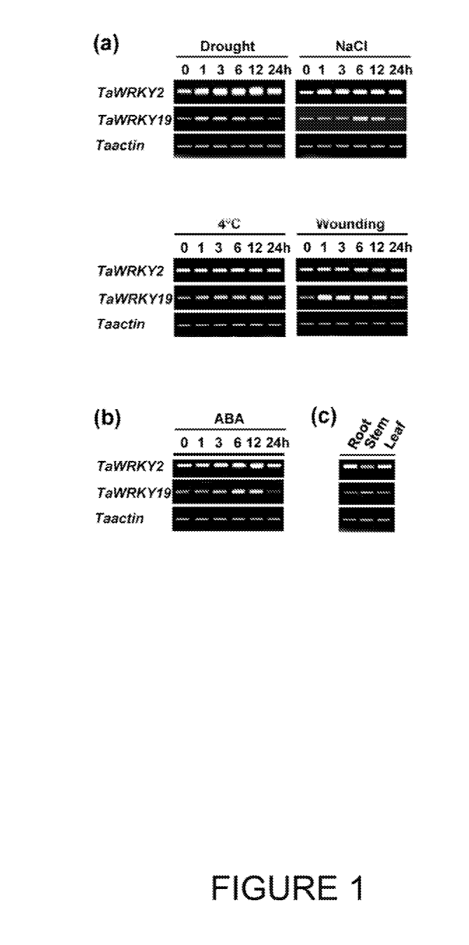

Among the TaWRKY genes identified, TaWRKY2 and 19 were induced by four or five of the aforementioned treatments (see FIGS. 1a and 1b). Under drought stress, TaWRKY2 was highly induced while TaWRKY19 was only mildly induced. Under salt stress, TaWRKY2 and TaWRKY19 were induced. In response to low temperature, TaWRKY19 was moderately induced but TaWRKY2 expression was not affected. Upon wounding, TaWRKY2 and TaWRKY19 expression was weakly or moderately induced. ABA induced expression of both genes.

The expression of the two genes was also examined in different organs of wheat plants. TaWRKY19 was weakly expressed in roots, stems and leaves. However, the expression level of TaWRKY2 was relatively high in roots and leaves but low in stems (see FIG. 1c).

### Example 3

### Overexpression of TaWRKY genes in transgenic Arabidopsis plants

A pGEM®-T-TaWRKY2 construct was separately digested with BamHI and SacI, and the resulting encoding sequences of TaWRKY2 was ligated into the plant expression vector pBIN121 driven by a constitutive CaMV 35S promoter, yielding the pBIN121-pBIN121-TaWRKY2 construct. In the same way, the full-length TaWRKY19 from the original pGEMO-T-TaWRKY19 construct was ligated to the SmaI/SacI site of the pBIN121 vector, generating the pBIN121-TaWRKY19 construct. The resulting constructs were confirmed by sequencing and then separately transformed into Agrobacterium tumefaciens GV3101 by electroporation following the transfection into Arabidopsis ecotype Columbia plants (Col-0) by the vacuum infiltration method described in e.g., Bechtold and Pelletier, Methods Mol. Biol. 82, 259-66 (1998).

The transgenic plants were selected on Murashige and Skoog agar (MS agar) plus 3% (w/v) sucrose and 50 mg/L kanamycin. Independent transgenic lines were created for TaWRKY2 (48 lines) and TaWRKY19 (8 lines). The expression of the TaWRKY transgene in each line was examined by RT-PCR using specific primers based on the full-length TaWRKY genes for all T₁ transgenic lines. Total RNA isolatation and RT-PCR analysis were carried out as described hereinabove. Col-0 was used as a negative control. An AtActin-7 gene was used as the internal control. For T₁ transgenic lines with high expression levels of TaWRKY genes, the transgene expression was further confirmed by Northern blot analysis. Total RNA (25 μg) was separated by 1.0% agarose gel containing formaldehyde, transferred onto Hybond N⁺ nylon membranes, and hybridized as described previously in Zhang et al., Theor. Appl. Genet, 99:1006-1011 (1999).

The BamHI/SacT and SmaI/SacI fragments containing the full-lengthTa WRKY gene from the original pGEM-T-TaWRKY constructs were labeled with α-³²P-dCTP by the random-priming method and used as probes for Northern blot analysis. 18S and 28S RNA were used as internal controls. Specific bands were visualized by using a Typhoon Trio scanner (Amersham Biosciences/GE Healthcare, USA). RT-PCR and Northern blot analyses were repeated twice with independent RNA samples. Transgenic lines overexpressing TaWRKY2 or TaWRKY19 were investigated in detail for their performance under different abiotic stresses.

### Example 4

### Performance of TaWRKY2 transgenic Arabidopsis plants

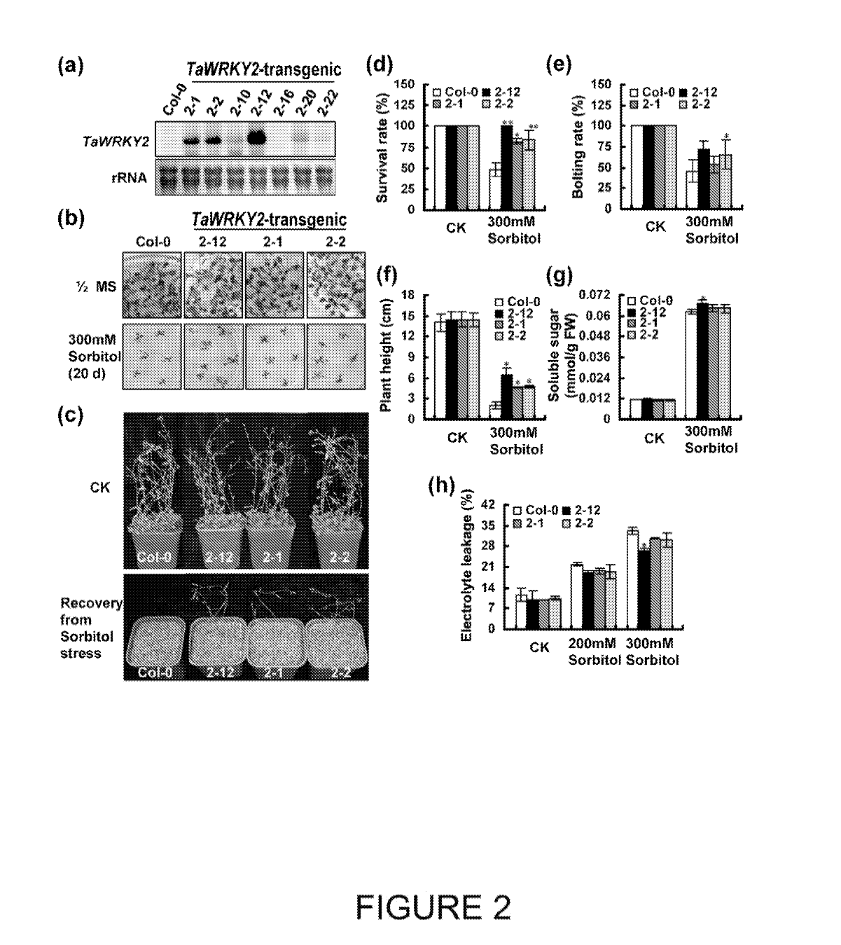

The homozygous T₃ or T₄ seeds of three transgenic Arabidopsis lines overexpressing TaWRKY2 (2-12, 2-1 and 2-2); see FIG. 2a) were examined for their performance under drought, salt and cold stresses.

Seeds of Col-0 and TaWRKY2 transgenic lines were surface-sterilized by 70% (v/v) ethanol for 3-5 min and 15% (v/v) bleach (Kao Corporation, Tokyo, Japan) for 10 minutes followed by five rinses with sterile water. The sterilized seeds were vernalized for 3 days at 4° C. in the dark and then grown on MS agar at pH 5.8 and subsequently on vermiculite at 23° C. under continuous light in growth chambers.

To evaluate drought and salt tolerance, 7-day-old seedlings of the Col-0 and transgenic lines growing on horizontally placed MS agar were carefully transferred onto 0.5×MS agar or 0.5×MS agar plus 300 mM sorbitol or 150 mM NaCl following horizontally placed growth. After sorbitol treatment for 20 days or NaCl treatment for 15 days, the resulting phenotypic changes were observed. The treated seedlings were transferred onto vermiculite for recovery from sorbitol treatment for 20 days and from NaCl treatment for 10 days and 25 days.

Seventeen-week-old seedlings of Col-0 and transgenic plants growing on MS agar were transferred into vermiculite for 4 days of cultivation at normal growth temperature and then used to evaluate tolerance of the transgenic plants. Three-week-old seedlings were moved into a cold room at 4° C. in light for 16 hours of cold acclimation and then into a temperature-regulated freezer at −4° C. in light for 4 days of freezing before 24 hours of deacclimation in a cold room at 4° C. Subsequently, the low temperature-treated seedlings were transferred to normal growth temperature for 20 days of recovery.

To evaluate the survival and growth of plants after recovery from sorbitol, NaCl and low temperature treatment, the resulting survival ratio, bolting ratio, and plant height were evaluated. The survival ratio represents the percentage of the number of the survival plants after recovery for indicated times over the number of the treated plants. The bolting ratio indicates the percentage of the number of the plants with bolts (≧5 mm) versus the number of the survival plants after recovery. The plant height denotes the average of the peak bolts of the plants with over 5-mm-length bolts after recovery.

To evaluate other indicators associated with plant responses to drought and salt stresses, 7-day-old seedlings of the Col-0 and transgenic TaWRKY2 plants were carefully transferred onto 0.5×MS agar and 0.5×MS agar plus various concentrations of sorbitol (200 mM and 300 mM) or NaCl (125 mM, and 150 mM) for 5 days. Rosette leaves from sorbitol-treated, NaCl-treated, and untreated controls were harvested to measure malondialdehyde (MDA) content, soluble sugar content, and electrolyte leakage.

MDA content and soluble sugar content were evaluated by a modified version of the thiobarbituric acid reactive substances (TBARS) assay described by Cui and Wang, Plant Soil Environ., 52:523-529 (2006). Leaves (about 0.4 g, M) were weighed, ground in liquid nitrogen with a pistil and mortar containing quartz yarn, and then added to 4 ml (V) 10% (w:v) trichloroacetic acid (TCA). The homogenate was centrifuged at 8 000×g for 20 min and 200 μl of the supernatant was mixed with 200 μl of 0.6% (w:v) thiobarbituric acid (TBA) in 10% TCA for reaction in boiled water for 15 min. The products were cooled to room temperature and centrifuged at 10,000×g for 10 min. The absorbance of the supernatant was examined at 450 nm (A₄₅₀), 532 nm (A₅₃₂), and 600 nm (A₆₀₀) with a UV-VIS spectrophotometer (Shimadzu, Japan). MDA content was evaluated as the formula: MDA (μmol/g FW)=[6.45×(A₅₃₂−A₆₀₀)−0.56×A₄₅₀]×V/M (FW represents fresh weight). Soluble sugar content was evaluated as the formula: soluble sugar (mmol/g FW)=11.71×A₄₅₀×V/M.

Electrolyte leakage of leaves was examined by using a DDS-11A conductivity detector (Kangyi), following the procedures as previously described by Cao et al., Plant Physiol., 143:707-719 (2007). The well-washed leaf discs from three seedlings were immersed in deionized water (final volume of 12 ml) subjected to vacuum infiltration for 20 minutes, and maintained in the water for 2 hours. The conductivities (C1) of the resulting solutions were determined. After the leaf segments in deionized water were boiled for 15 min, the corresponding solutions were cooled to room temperature and their conductivities (C2) were yielded. The percentage of C1 divided by C2 (C1/C2) were denoted as the electrolyte leakage.

To evaluate other indicators associated with plant responses to cold stress, three-week-old seedlings of the Col-0 and transgenic TaWRKY2 plants were acclimated at 4° C. for 16 hours in a cold room and then exposed to −4° C. for 4 days in a temperature-regulated freezer. Rosette leaves from cold-stressed and untreated controls were harvested to measure MDA content, soluble sugar content, and electrolyte leakage as described above.

After treatment with 300 mM sorbitol for 20 days, the transgenic plants remained green while most leaves of the Col-0 plants turned yellow (see FIG. 2b). The plate-grown plants were subsequently transferred into pots containing vermiculite for recovery under normal conditions. After 20 days the transgenic plants showed better survival and growth than the Col-0 plants as judged from the survival rate, bolting rate and plant height (see FIGS. 2c, d, e and f).

In drought stress-treated plants, the soluble sugar level was significantly upregulated in the 2-12 line but only slightly increased in the other two lines (see FIG. 2g). The relative electrolyte leakage was significantly reduced in the 2-12 line under 300 mM sorbitol treatment but only slightly decreased in the other lines at the same treatment. At 200 mM sorbitol, all the three lines showed slight reductions in the relative electrolyte leakage (see FIG. 2h). Under normal conditions, the Col-0 and the transgenic plants grew well and showed no significant difference in phenotypes and physiological parameters.

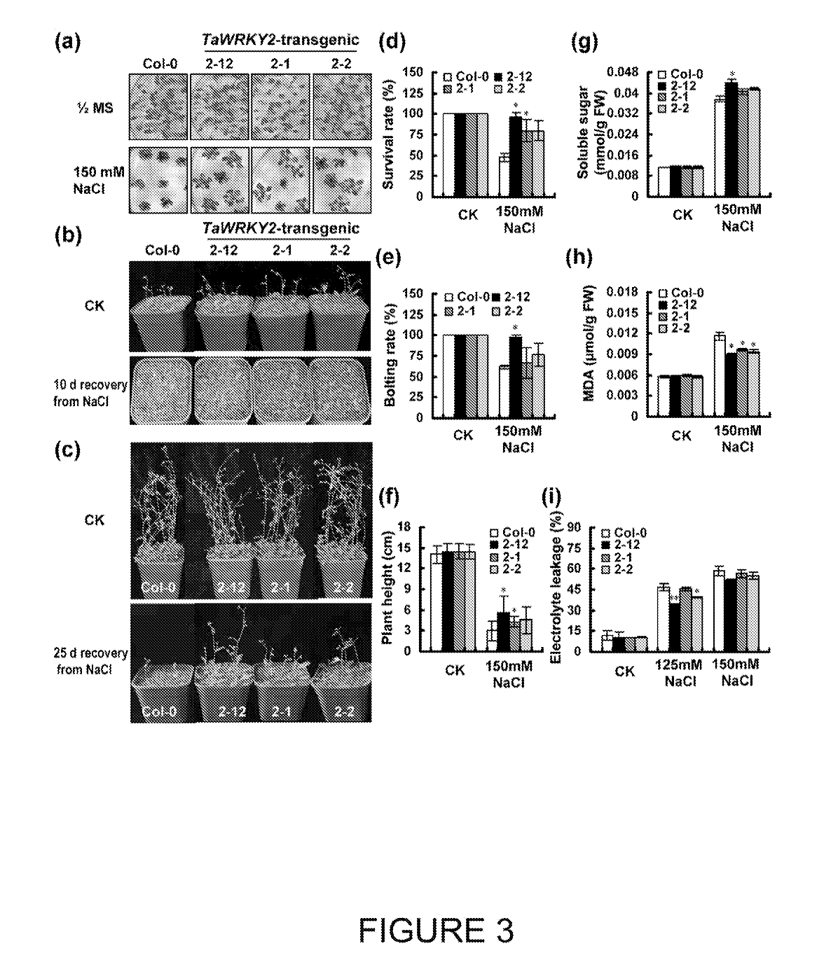

In salt-stressed consitions, rosette leaves of the Col-0 plants showed severe epinasty whereas most of the transgenic plants were only moderately affected (see FIG. 3a). Salt-treated plants were subsequently transferred into pots containing vermiculite and allowed to recover under normal conditions. After recovery for 10 days, more than 79% of the transgenic plants survived whereas less than 50% of the Col-0 plants survived (see FIGS. 3b, d). After recovery for 25 days, the salt-treated transgenic lines exhibited a higher bolting rate than the Col-0 plants (see FIGS. 3c, e). The inflorescence of the salt-treated transgenic plants was also taller than that of the stressed Col-0 plants (see FIG. 3f). Soluble sugar levels were substantially enhanced in salt-stressed transgenic plants compared to the stressed Col-0 plants (see FIG. 3g).

On the contrary, the MDA levels and the relative electrolyte leakage were reduced in the Ta WRKY2-overexpressing plants after salt stress in comparison with the salt-treated Col-0 plants (see FIGS. 3h, i). No significant difference in phenotypes or physiological parameters was observed between the Col-0 and TaWRKY2-transgenic plants under normal growth conditions (see FIGS. 3a-i).

These results indicate that TaWRKY2 enhances plant tolerance to osmotic stress, such as that induced in drought conditions, and enhances plant tolerance to salt stress.

### Example 5

### Performance of TaWRKY19 transgenic Arabidopsis plants

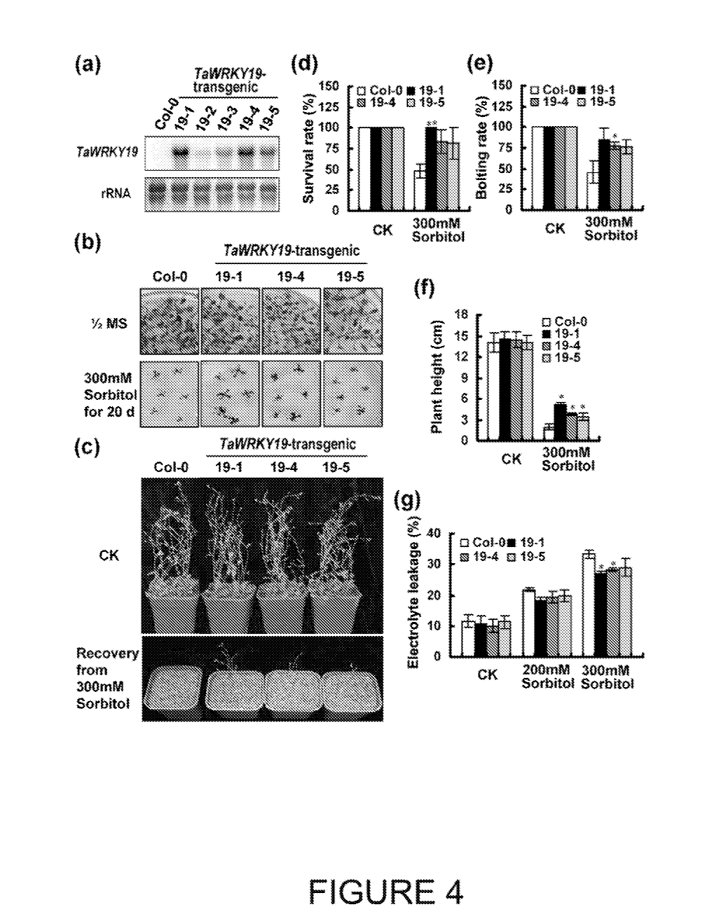

Three independent transgenic lines overexpressing TaWRKY19 (19-1, 19-4, and 19-5; see FIG. 4a), were also examined for their performance under cold, salt and drought stresses as described in Example 4.

After 300 mM sorbitol treatment for 20 days, the transgenic plants exhibited more green leaves than the control Col-0 plants (see FIG. 4b). After transfer of the sorbitol-treated seedlings into vermiculite and recovery for 20 days, the transgenic lines showed better growth than Col-0 controls as demonstrated by the higher survival rate, higher bolting rate and taller influorescence (see FIGS. 4c, d, e and f). The relative electrolyte leakage was also reduced in the TaWRKY19-overexpressing plants after sorbitol treatment compared to the level in Col-0 plants (see FIG. 4g). Under normal conditions, no significant difference was observed between the Col-0 and TaWRKY19-transgenic plants in plant growth and electrolyte leakage (see FIG. 4).

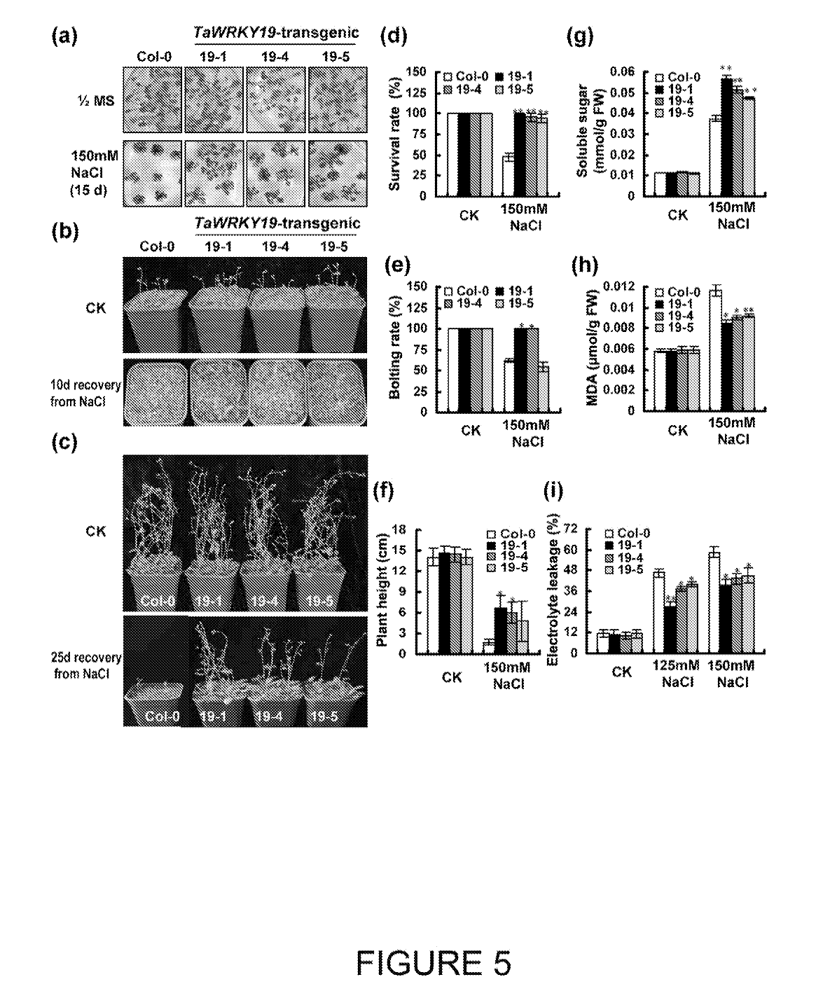

After treatment with 150 mM NaCl for 15 days, transgenic plants showed better growth than the control plants (see FIG. 5a). After recovering for 10 days, more than 94% of the salt-treated transgenic plants survived while only 47% of the salt-treated control plants survived (see FIGS. 5b, d). After recovery for 25 days, transgenic lines 19-1 and 19-4 had significantly higher bolting rates than controls and transgenic line 19-5 (see FIGS. 5c, e). The inflorescence of the transgenic plants was also taller than that of the control plants (see FIGS. 5c, f). Soluble sugar levels were significantly higher whereas both the MDA and electrolyte leakage levels were lower in salt-stressed transgenic plants than those in salt-stressed control plants (see FIGS. 5g, h, i).

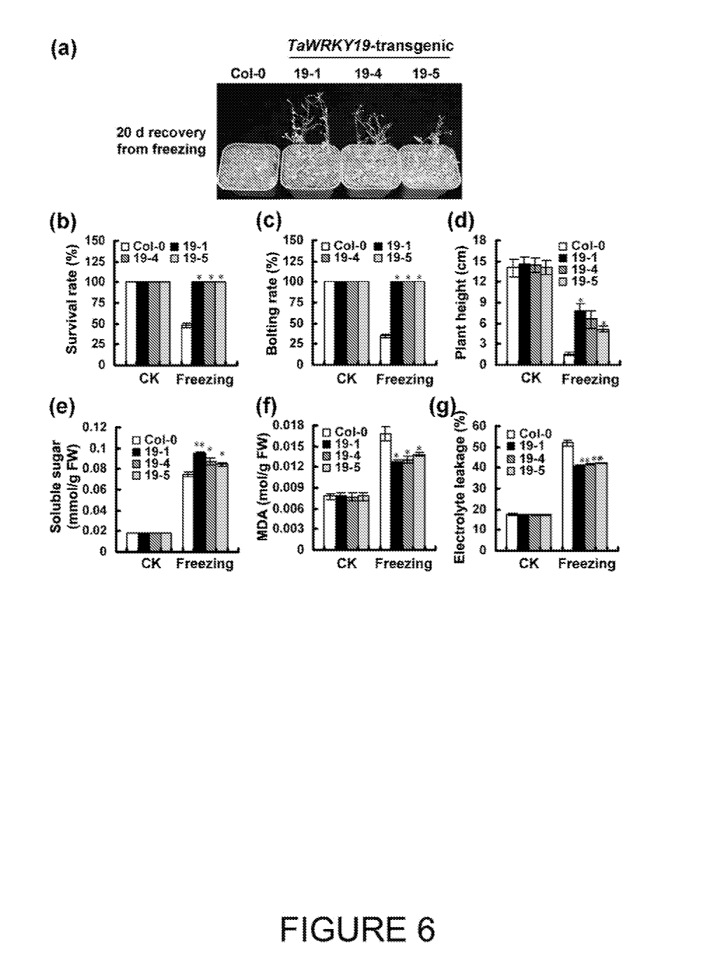

Three-week-old seedlings growing in vermiculite were exposed to freezing temperature and then allowed to recover under normal growth conditions. After treatment, all TaWRKY19-transgenic plants were alive and bolted (see FIGS. 6a, b, c). On the contrary, less than half of the control plants survived and only ˜35% of the survived control plants bolted (see FIGS. 6a, b, c). In the bolted plants, the mean plant heights of the transgenic plants were greater than that of the control plants (see FIG. 6d). Cold-treated transgenic plants also had higher levels of soluble sugars but lower levels of MDA and relative electrolyte leakage compared to the freezing-treated control plants (see FIGS. 6e, f, g). Under normal growth conditions, all transgenic and control plants grew well and showed no significant difference in phenotype and physiological parameters (see FIG. 6).

These results indicate that TaWRKY19 enhances plant tolerance to drought, salt and cold stresses.

### Example 6

### Subcellular Localization of TaWRKY Proteins

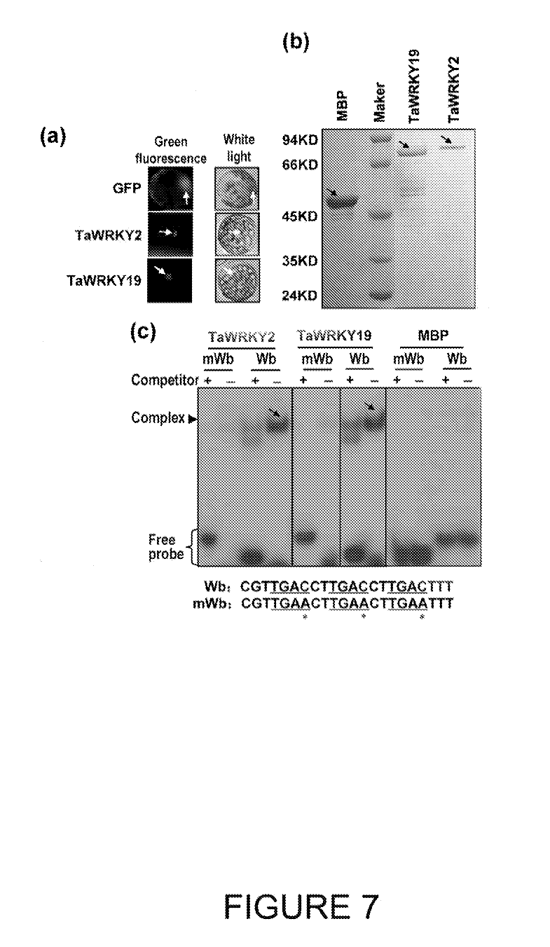

For subcellular localization of TaWRKY proteins, the complete coding sequences of TaWRKY2 and TaWRKY19 were obtained by PCR with specific primers, digested with the restriction enzymes BamHI/SalI and XhoI/SpeI, and then fused to the 5′ or 3′-terminus of a green fluorescent protein (GFP) gene in a transient expression vector to generate pUC-pUC-TaWRKY2-GFP and pUC-GFP-TaWRKY19 constructs under the control of a constitutive CaMV 35S promoter. The two constructs and the positive control pUC-GFP plasmid were separately transfected into Arabidopsis protoplasts prepared from suspended cells. GFP fluorescence was observed under a confocal microscope. All the TaWRKY-GFP fusion proteins were restricted to the cell nucleus while the control GFP protein was observed in the cytoplasm (see FIG. 7a). These results indicate that both TaWRKYs are nuclear proteins.

### Example 7

### Transcription Activation of TaWRKY Proteins

TaWRKY2 and TaWRKY19 were also investigated for their transcription activation ability in both yeast and Arabidopsis protoplast assay systems. In the yeast assay system, each of the TaWRKY genes was fused to the DNA sequence encoding the GAL4 DNA-binding domain in the plasmid GAL4 DBD. The resultant pBD-TaWRKY fusion plasmids were transfected into YRG-2 yeast cells, which contain integrated reporter genes lacZ and HIS3 under the control of the GAL4 upstream activating sequence (UAS).

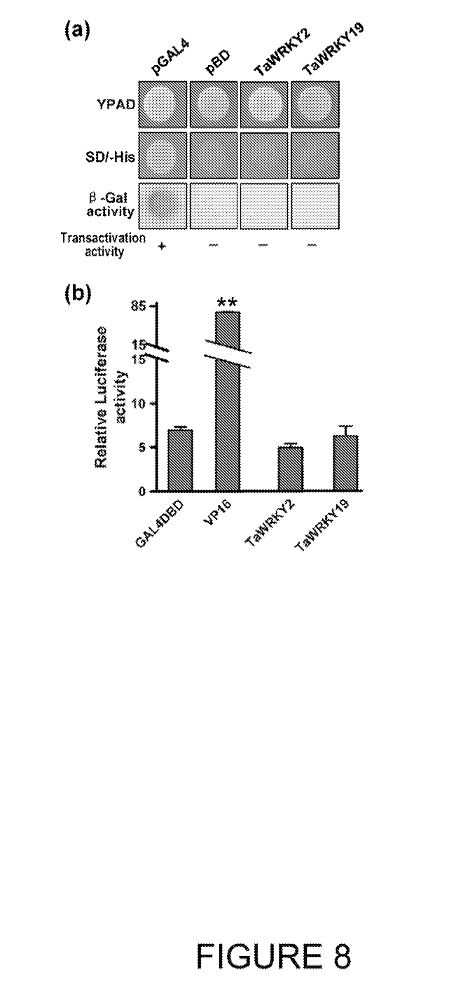

As shown in FIG. 8a, yeast cells containing pBD-TaWRKY2, pBD-TaWRKY19 or the negative control plasmid pBD could not grow and did not show blue color in the presence of X-Gal. All these transformants grew well on normal YPAD medium. These results indicate that TaWRKY2 and TaWRKY19 do not possess transcriptional activation activity (at least in this yeast system).

A dual-luciferase reporter assay system (Promega, USA) was used to examine the transcriptional activation activity of the TaWRKY proteins in Arabidopsis protoplasts. The effector plasmids pBD-TaWRKYs and the reporter plasmid expressing firefly luciferase (LUC) were co-transfected into Arabidopsis protoplasts, and the relative LUC activity was determined. As shown in FIG. 8b, TaWRKY2 and TaWRKY19 had less LUC activity than the GAL4 DBD negative control. These results indicate that TaWRKY2 and TaWRKY19 appears to have no transcriptional activation ability in a protoplast assay.

### Example 8

### DNA-Binding Specificity of TaWRKY Proteins

The ability of TaWRKY2 and TaWRKY19 to bind the W box (TTGACC/T) was also investigated. Truncated coding sequences of TaWRKY2 and TaWRKY19 containing two complete WRKY domains were PCR-amplified using specific primers containing BamHI and SalI sites and EcoRI and SalI sites and then fused into pMAL-c2X vector (New England Biolabs, USA) which includes a maltose binding protein (MBP) encoding gene. The TaWRKY2 and -19 truncations have two WRKY domains, and range from amino acid position 181 to 468 and 193 to 468 respectively. These two expressed proteins have expected molecular weights of 74 and 73 KD, respectively (see FIG. 7b).

The MBP-fused TaWRKY truncation proteins were expressed in Escherichia coli strain TB1 and purified by using an amylose resin affinity column. Expression of the fusion proteins was induced by 0.15 mM isopropyl-D-thiogalactopyranoside (IPTG) by shaking the cells a 180 rpm for 8 hours at 25° C. TaWRKY proteins were separated by SDS-PAGE on a 10% polyacrylamide gel for and stained with coomassie brilliant blue R-250. MBP was also purified as a control.

To analyze the binding specificity of TaWRKY proteins to a W box, 2 pairs of synthetic single-stranded oligonucleotides Wb and mWb, each containing three tandem TTGAC repeats and the mutated TTGAA sequence, were annealed in 50 mM NaCl by heating at 70° C. for 5 minutes followed by cooling slowly to room temperature to generate doublestrand oligonucleotides. The doublestrand oligonucleotides were end-labelled by [γ-³²P]-dATP (Amersham Pharmacia) and T4 polynucleotide kinase (Promega) following the manufacturer's manual, and the ³²P-labeled oligonucleotides were purified by using spin-column chromatography (G-25 Sephadex column, Amersham Pharmacia).

The binding reaction was incubated at room temperature for 20 min in 5 μl of mixture containing 2-4 μg purified protein, 1 μl of [γ-³²P]-labelled DNA fragment and 2 μl of gel shift binding 5× Buffer [20% glycerol, 5 mM MgCl, 5 mM ZnSO₄, 2.5 mM ethylenediaminetetraacetic acid (EDTA), 2.5 mM dithiothreitol (DTT), 250 mM NaCl, 50 mM tris(hydroxymethyl)aminomethane (Tris)-HCl, 0.25 mg/mL poly(dI-dC)poly(dI-dC), pH 7.5]. The protein/DNA complexes were separated with 6% non-denaturing polyacrylamide gels in 0.5×TBE buffer (0.1 M Tris, 90 mM boric acid, 1 mM EDTA) at 200 V for 35 minutes until the bromophenol blue dye traveled ⅗ths of the length of the gel.

Gels were subsequently transferred onto filter paper, dried at room temperature and exposed to X-ray film at −70° C. with an intensifying screen. Specific bands were visualized using a Typhoon Trio scanner (Amersham Biosciences/GE Healthcare, USA). Probe binding specificity was assessed by attenuation of the retarded bands with equivalent labelled mutant probe and by competition assays using up to a 10-fold excess of unlabeled probe in binding reaction.

Both proteins formed a complex with the labeled Wb element (see FIG. 7c). The intensities of the retarded bands were significantly reduced when non-labeled competitor probes were included, indicating that both TaWRKY proteins can specifically bind to the W box sequence.

Both proteins were also tested for their ability to bind the mutant element mWb. Neither protein exhibited significant binding to the mutant (FIG. 7c). MBP showed no binding to either Wb or mWb elements. These results indicate that both TaWRKY proteins can specifically bind to the Wb element.

### Example 9

### Altered Gene Expression in the Ta WRKY-Transgenic Plants

Total RNA was isolated from 12-day-old seedlings of the Col-0 and transgenic plants and the expression of stress-related genes DREB2A, RD29A, RD29, COR15A, STZ and COR6.6 was investigated in Ta WRKY-transgenic plants by RT-PCR and further confirmed by Northern blot analysis. The specific primers for the various stress-related genes were based on Arabidopsis gene sequences downloaded from www.arabidopsis.org.

To test the binding specificity of TaWRKY proteins to the promoter regions of the regulated genes, the presence of putative WRKY-binding W box elements were further investigated in 1.5 kb promoter regions of the Ta WRKY-regulated genes. The 1.5 kb promoter sequences were obtained from the Arabidopsis Biological Resource Center (ABRC). Following these promoter sequences, forward and reverse single strand oligonucleotides corresponding to particular DNA fragments were synthesized, annealed and then end-labelled by [γ-³²P]-dATP (Amersham Pharmacia) as probes.

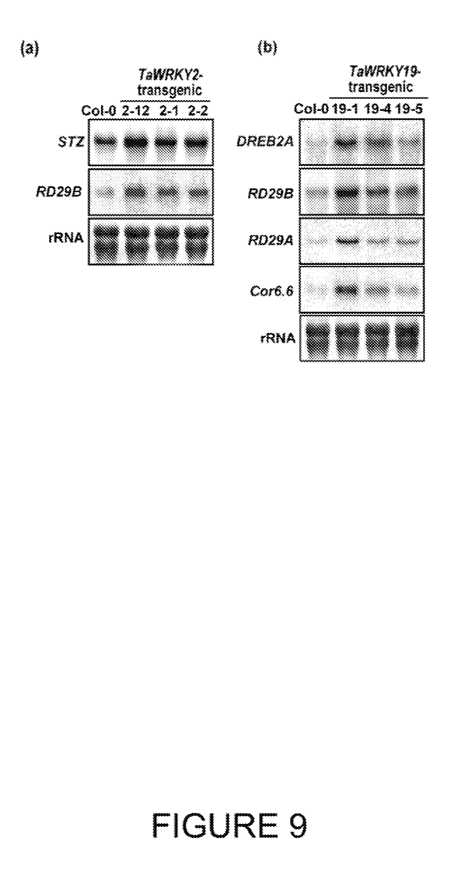

The expression of the RD29B and STZ genes was enhanced in the TaWRKY2-transgenic plants, indicating that TaWRKY2 may function in stress tolerance at least through regulation of the two genes (see FIG. 9a). In Ta WRKY19-transgenic plants, the DREB2A, RD29B, Cor6.6, and RD29A genes were expressed in a higher level than those in the Col-0 plants (see FIG. 9b). These results suggest that both TaWRKY genes improve stress tolerance through regulation of different downstream genes.

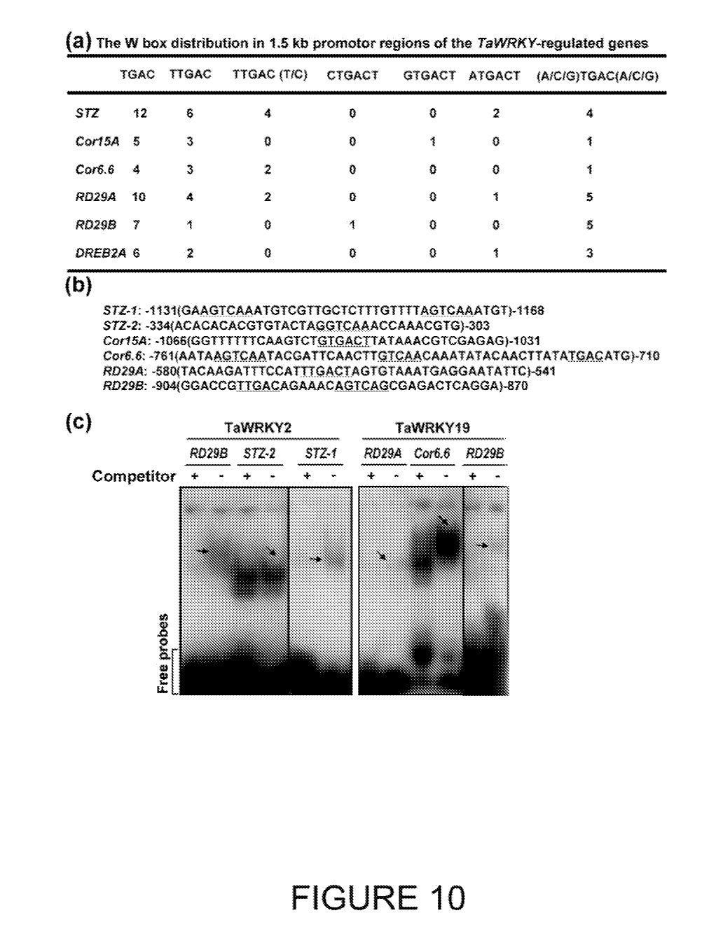

The distribution frequency of each element was different among different genes and the predicted elements with different flanking sequences were noted (see FIG. 10a). Considering that the WRKY protein SUSBIBA2 can bind to the atypical W box sequence GTGACT in the barley isol promoter, both the typical W box TTGAC(T/C) and the atypical W box GTGAC in the 1.5 kb promoter regions of the STZ, Cor6.6, RD29A, and Cor15A genes were synthesized for a gel shift assay (see FIG. 10b). These two W-boxes were absent in the 1.5 kb promotor regions of the RD29B and DREB2A genes (see FIG. 11a). However, because the putative TTGACA element and CTGACT element in complementary strand were found in the −898 by to −882 by region of RD29B with spacing of 5 bp, the corresponding DNA fragment was also synthesized for further analysis (see FIG. 11b).

TaWRKY2 showed moderate specific binding to the elements from RD29B and STZ-1, and had nonspecific binding to the element from STZ-2 (see FIG. 10c). TaWRKY19 exhibited strong specific binding to the element from Cor6.6 gene, but only very weak specific binding to the elements from RD29A and RD29B genes (see FIG. 10c). These results suggest that TaWRKY2 and TaWRKY19 totally regulate at least four of the six downstream genes through direct binding to the elements in their promoters.

The disclosure of every patent, patent application, and publication cited herein is hereby incorporated herein by reference in its entirety.

While this invention has been disclosed with reference to specific embodiments, it is apparent that other embodiments and variations of this invention can be devised by others skilled in the art without departing from the true spirit and scope of the invention. The appended claims include all such embodiments and equivalent variations.

1.-4. (canceled)

5. A transgenic plant comprising in its genome an isolated polynucleotide encoding a WRKY polypeptide, wherein the polynucleotide is selected from the group consisting of:
(a) a nucleic acid comprising a nucleotide sequence of any one of SEQ ID NOs: 1 and 3;
(b) a nucleic acid comprising a nucleotide sequence at least 95% identical to (a);
c) a nucleic acid comprising a nucleotide sequence that specifically hybridizes to the complement of (a) under stringent hybridization conditions comprising 50% formamide and 1 mg of heparin overnight at 40° C. and wash conditions comprising 0.2×SSC at 65° C. for 15 minutes;
(d) a nucleic acid comprising an open reading frame encoding a WRKY polypeptide comprising an amino acid sequence of any one of SEQ ID NOs: 2 and 4;
(e) a nucleic acid comprising an open reading frame encoding a WRKY polypeptide comprising an amino acid sequence at least 95% identical to (d) and further comprising at least one WRKY domain; and
(f) a nucleic acid comprising a nucleotide sequence that is the complement of any one of (a)-(e).

6. The transgenic plant of claim 5, wherein the plant is a monocot.

7. The transgenic plant of claim 5, wherein the plant is a dicot.

8. The transgenic plant of claim 5, wherein the transgenic plant is selected from the group consisting of maize, wheat, barley, rye, sweet potato, bean, pea, chicory, lettuce, cabbage, cauliflower, broccoli, turnip, radish, spinach, asparagus, onion, garlic, pepper, celery, squash, pumpkin, hemp, zucchini, apple, pear, quince, melon, plum, cherry, peach, nectarine, apricot, strawberry, grape, raspberry, blackberry, pineapple, avocado, papaya, mango, banana, soybean, tomato, sorghum, sugarcane, sugar beet, sunflower, rapeseed, clover, tobacco, carrot, cotton, alfalfa, rice, potato, eggplant, cucumber, and Arabidopsis.

9. (canceled)

10. A method for producing the transgenic plant of claim 5 comprising:
(a) introducing an isolated polynucleotide encoding a WRKY polypeptide into a plant cell to produce a transgenic plant cell, wherein the polynucleotide is selected from the group consisting of:
(i) a nucleic acid comprising a nucleotide sequence of any one of SEQ ID NOs: 1 and 3;
(ii) a nucleic acid comprising a nucleotide sequence at least 95% identical to (i);
(iii) a nucleic acid comprising a nucleotide sequence that specifically hybridizes to the complement of (i) under stringent hybridization conditions comprising 50% formamide and 1 mg of heparin overnight at 40° C. and wash conditions comprising 0.2×SSC at 65° C. for 15 minutes;
(iv) a nucleic acid comprising an open reading frame encoding a WRKY polypeptide comprising an amino acid sequence of any one of SEQ ID NOs: 2 and 4;
(v) a nucleic acid comprising an open reading frame encoding a WRKY polypeptide comprising an amino acid sequence at least 95% identical to (iv) and further comprising at least one WRKY domain; and
(vi) a nucleic acid comprising a nucleotide sequence that is the complement of any one of (i)-(v); and
(b) regenerating a transgenic plant from the transgenic plant cell of (a), wherein the transgenic plant comprises in its genome the isolated polynucleotide encoding the WRKY polypeptide.

11. A method for producing a transgenic plant comprising crossing a transgenic plant according to claim 5 with a non-transgenic plant.

12. A transgenic plant produced by the method according to claim 10.

13. A method of altering a trait in a plant comprising expressing an isolated polynucleotide encoding a WRKY polypeptide in the plant, wherein the polynucleotide is selected from the group consisting of:
(a) a nucleic acid comprising a nucleotide sequence of any one of SEQ ID NOs: 1 and 3;
(b) a nucleic acid comprising a nucleotide sequence at least 95% identical to (a);
(c) a nucleic acid comprising a nucleotide sequence that specifically hybridizes to the complement of (a) under stringent hybridization conditions comprising 50% formamide and 1 mg of heparin overnight at 40° C. and wash conditions comprising 0.2×SSC at 65° C. for 15 minutes;
(d) a nucleic acid comprising an open reading frame encoding a WRKY polypeptide comprising an amino acid sequence of any one of SEQ ID NOs: 2 and 4;
(e) a nucleic acid comprising an open reading frame encoding a WRKY polypeptide comprising an amino acid sequence at least 95% identical to (d) and further comprising at least one WRKY domain; and
(f) a nucleic acid comprising a nucleotide sequence that is the complement of any one of (a)-(e).

14. The method of claim 13, wherein the trait is selected from the group consisting of drought tolerance, salt tolerance and cold tolerance.

15. The method of claim 14, wherein the isolated polynucleotide encoding the WRKY polypeptide comprises SEQ ID NO: 1 and the trait is drought tolerance.

16. The method of claim 14, wherein the isolated polynucleotide encoding the WRKY polypeptide comprises SEQ ID NO: 1 and the trait is salt tolerance.

17. The method of claim 14, wherein the isolated polynucleotide encoding the WRKY polypeptide comprises SEQ ID NO: 3.

18. A transgenic plant produced by the method according to claim 13.

19. A method for producing a transgenic plant comprising crossing the plant according to claim 18 with a non-transgenic plant.

20. The method of claim 13, wherein the method comprises:
(a) introducing the isolated polynucleotide encoding a WRKY polypeptide into a plant cell to produce a transgenic plant cell; and
(b) regenerating a transgenic plant from the transgenic plant cell of (a), wherein the transgenic plant comprises in its genome the isolated polynucleotide encoding the WRKY polypeptide.

21. A transgenic plant produced by the method according to claim 19.

22. A transgenic seed produced from the transgenic plant of claim 5, wherein the seed comprises in its genome the isolated polynucleotide encoding the WRKY polypeptide.

23. A transgenic seed produced from the transgenic plant of claim 12, wherein the seed comprises in its genome the isolated polynucleotide encoding the WRKY polypeptide.

24. A transgenic seed produced from the transgenic plant of claim 18, wherein the seed comprises in its genome the isolated polynucleotide encoding the WRKY polypeptide.

25. A transgenic seed produced from the transgenic plant of claim 21, wherein the seed comprises in its genome the isolated polynucleotide encoding the WRKY polypeptide.
